(19) United States
(12) Patent Application Publication
(10) Pub. No.: US 2019/0239793 A1
(43) Pub. Date: Aug. 8, 2019

(54) STATE ACQUISITION COMPUTER, STATE ACQUISITION METHOD, AND INFORMATION PROCESSING SYSTEM

(71) Applicants: Sharp Kabushiki Kaisha, Sakai City, Osaka (JP); Osaka Prefecture University Public Corporation, Sakai-shi, Osaka (JP)

(72) Inventors: Tetsuya HAYASHI, Sakai City (JP); Hiroshi SAKAYA, Sakai City (JP); Azusa NAKANO, Sakai City (JP); Syunsuke SHIMAMURA, Sakai-shi (JP); Terumasa SHIMADA, Sakai-shi (JP)

(21) Appl. No.: 16/333,285

(22) PCT Filed: Jan. 26, 2017
(86) PCT No.: PCT/JP2017/002738

§ 371 Date: Mar. 14, 2019

(30) Foreign Application Priority Data
Sep. 20, 2016 (JP) 2016-182682

(51) Int. Cl. A61B 5/16; A61B 5/044; A61B 5/0468; A61B 5/00

(52) U.S. Cl. CPC A61B 5/165; A61B 5/044; A61B 2503/40; A61B 5/7235; A61B 5/0468

(57) ABSTRACT

A state acquisition computer is provided that includes an interface configured to acquire data for indicating an inter-beat interval of an animal, and a processor configured to convert a Poincare plot of inter-beat intervals into a prescribed coordinate system, and based on a standard deviation of the Poincare plot after conversion, acquire information indicating a psychological state or a physical state of the animal.

### TECHNICAL FIELD

The disclosure below relates to technology for acquiring a psychological state or a physical state of an animal, and especially relates to a state acquisition computer, a state acquisition method, and an information processing system for acquiring a psychological state or a physical state of an animal from an inter-beat interval.

### BACKGROUND ART

Conventionally, technology for acquiring a psychological or physical state of an animal has been known. For example, a pulse wave diagnostic apparatus and a pulse wave diagnostic apparatus control method are disclosed in JP 5160586 B (PTL 1). According to PTL 1, a pulse wave diagnostic apparatus is provided that includes a photoelectric pulse wave detecting unit configured to receive light transmitted through an artery or light scattered at the artery to detect a pulse wave, a pulse wave amplitude Poincare calculation unit configured to calculate a pulse wave amplitude for each pulse of a pulse wave detected by the photoelectric pulse wave detecting unit to calculate a point of the pulse wave amplitude on an orthogonal coordinate plane formed by the two sequentially calculated pulse wave amplitudes as Poincare coordinates for each pulse, and a pulse wave amplitude distribution calculation unit configured to calculate a variation width between a maximum point and a minimum point of the Poincare coordinates of the pulse wave amplitudes calculated by the pulse wave amplitude Poincare calculation unit, in which the pulse wave amplitude distribution calculation unit calculates a variation width between a maximum point and a minimum point in a range defined by two straight lines represented by Y=X+a, and Y=X−a on the Poincare coordinates of the pulse wave amplitudes calculated by the pulse wave amplitude Poincare calculation unit.

### CITATION LIST

### Patent Literature

PTL 1: JP 5160586 B

### SUMMARY OF INVENTION

### Technical Problem

Technology has been required in which a psychological or physical state of an animal can be recognized more precisely or efficiently than the conventional technology. The present disclosure is made to solve the problem, and an object of the present disclosure is to provide a state acquisition computer, a state acquisition method, and an information processing system that can recognize a psychological state or a physical state of an animal more precisely or efficiently than the conventional technology.

### Solution to Problem

According to an aspect of the present invention, a state acquisition computer is provided that includes an interface configured to acquire data for indicating an inter-beat interval of an animal, and a processor configured to convert a Poincare plot of inter-beat intervals into a prescribed coordinate system, and based on a standard deviation of the Poincare plot after conversion, acquire information indicating a psychological state or a physical state of the animal.

Preferably, the processor is configured to acquire the information indicating the psychological state or the physical state of the animal, based on a product of standard deviations of at least two axes of the Poincare plot.

According to another aspect of the present invention, a state acquisition computer is provided that includes an interface configured to acquire data for indicating an inter-beat interval of an animal, and a processor configured to acquire information indicating a psychological state or a physical state of the animal, based on an average of distances between two sequential plots of a Poincare plot of inter-beat intervals.

According to another aspect of the present invention, a state acquisition computer is provided that includes an interface configured to acquire data for indicating an inter-beat interval of an animal, and a processor configured to acquire information indicating a psychological state or a physical state of the animal, based on an amount or a percentage of plots in a center portion of a Poincare plot of inter-beat intervals.

Preferably, the state acquisition computer further includes a memory configured to store a numerical value range corresponding to each of a plurality of types of psychological states or physical states. The processor is configured to identify a psychological state or a physical state of the animal by referring to the numerical value range, based on the standard deviation, the average, or the amount or percentage of the plots.

According to another aspect of the present invention, a state acquisition computer is provided that includes an interface configured to acquire data for indicating an inter-beat interval of an animal, and a processor configured to acquire information indicating a psychological state or a physical state of the animal by generating a histogram of the inter-beat intervals.

According to another aspect of the present invention, a state acquisition computer is provided that includes an interface configured to acquire data for indicating an inter-beat interval of an animal, and a processor configured to acquire information indicating a psychological state or a physical state of the animal by generating a trajectory of a Poincare plot of the inter-beat intervals.

Preferably, the state acquisition computer further includes a memory configured to store data related to a histogram or a trajectory of a Poincare plot corresponding to each of a plurality of types of psychological states or physical states. The processor is configured to identify a psychological state or a physical state of an animal by referring to the data, based on the histogram generated or the trajectory of the Poincare plot generated.

Preferably, the processor is configured to acquire the psychological state or the physical state of the animal by using a plurality of methods among the above methods for acquiring the information indicating the psychological state or the physical state of the animal.

Preferably, an animal being a target has a respiratory sinus arrhythmia.

According to another aspect of the present invention, a state acquisition method of a psychological state or a physical state of an animal by using a computer including a processor is provided. The state acquisition method is provided that includes a step for acquiring data for indicating an inter-beat interval of an animal, a step for converting a Poincare plot of inter-beat intervals into a prescribed coordinate system, a step for calculating a standard deviation of the Poincare plot after conversion, and a step for acquiring information indicating a psychological state or a physical state of the animal, based on the standard deviation.

According to another aspect of the present invention, a state acquisition method of a psychological state or a physical state of an animal by using a computer including a processor is provided. The state acquisition method includes a step for acquiring data for indicating an inter-beat interval of an animal, a step for calculating an average of distances between two sequential plots of a Poincare plot of the inter-beat intervals, and a step for acquiring information indicating a psychological state or a physical state of the animal, based on the average.

According to another aspect of the present invention, a state acquisition method of a psychological state or a physical state of an animal by using a computer including a processor is provided. The state acquisition method includes a step for acquiring data for indicating an inter-beat interval of an animal, a step for generating a histogram of the inter-beat intervals, and a step for acquiring information indicating a psychological state or a physical state of the animal, based on the histogram.

According to another aspect of the present invention, a state acquisition method of a psychological state or a physical state of an animal by using a computer including a processor is provided. The state acquisition method includes a step for acquiring data for indicating an inter-beat interval of an animal, a step for generating a trajectory of a Poincare plot of the inter-beat intervals, and a step for acquiring information indicating a psychological state or a physical state of the animal, based on the trajectory.

According to another aspect of the present invention, a state acquisition method of a psychological state or a physical state of an animal by using a computer including a processor is provided. The state acquisition method includes a step for acquiring data for indicating an inter-beat interval of an animal, and a step for acquiring information indicating a psychological state or a physical state of the animal, based on an amount of plots in a center portion of a Poincare plot of inter-beat intervals.

According to another aspect of the present invention, an information processing system is provided that includes an output device, a sensor configured to detect a pulsation of an animal, and a computer configured to acquire data for indicating an inter-beat interval of the animal from the sensor, convert a Poincare plot of inter-beat intervals into a prescribed coordinate system, acquire information indicating a psychological state or a physical state of the animal based on a standard deviation of the Poincare plot after conversion, and cause the output device to output the information.

### Advantage Effects of Invention

As described above, the present disclosure provides a state acquisition computer, a state acquisition method, and an information processing system that can recognize a psychological state or a physical state of an animal more precisely or efficiently than the conventional technology.

### BRIEF DESCRIPTION OF DRAWINGS

FIG. 1 is a diagram showing an entire configuration of a state acquisition system 1 according to a first embodiment.

FIG. 2 is a diagram showing a functional configuration of the state acquisition system 1 according to the first embodiment.

FIG. 3 is a flowchart showing a processing procedure of the state acquisition system 1 according to the first embodiment.

FIG. 4 is a diagram showing a table indicating correspondence relation between an inter-beat interval R-R (n) and the following inter-beat interval R-R (n+1) according to the first embodiment.

FIG. 5 is a conceptual diagram showing conversion from a table 321A indicating correspondence relation between an inter-beat interval R-R (n) and the following inter-beat interval R-R (n+1) into axes in a Y=X direction and a direction perpendicular to the Y=X direction according to the first embodiment.

FIG. 6 is a table showing standards of standard deviations related to a Y=X axis and an axis perpendicular to the Y=X axis for each of psychological states or physical states of a dog according to the first embodiment.

FIG. 7 is a diagram showing an entire configuration of the state acquisition system 1 including a server according to the first embodiment.

FIG. 8 is a Poincare plot diagram of a dog in a state of excitement according to the first embodiment.

FIG. 9 is a Poincare plot diagram of a dog in a normal state in which breathing of the dog is stable according to the first embodiment.

FIG. 10 is a Poincare plot diagram of a dog in a normal state according to the first embodiment.

FIG. 11 is a Poincare plot diagram in a rest state of a dog according to the first embodiment.

FIG. 12 shows a processing procedure of the state acquisition system 1 according to a second embodiment.

FIG. 13 is a table showing standards of standard deviations related to a Y=X axis and an axis perpendicular to the Y=X axis, a product of the standard deviations, and a ratio of the standard deviations for each of psychological states or physical states of a dog according to the second embodiment.

FIG. 14 shows a processing procedure of the state acquisition system 1 according to a third embodiment.

FIG. 15 is a flowchart showing a processing procedure of the state acquisition system 1 according to a fourth embodiment.

FIG. 16 is a diagram showing a distance table 321B according to the fourth embodiment.

FIG. 17 is a table showing a standard of an average of distances for each of psychological states or physical states of a dog according to the fourth embodiment.

FIG. 18 is a diagram showing a functional configuration of the state acquisition system 1 according to a fifth embodiment.

FIG. 19 is a flowchart showing a processing procedure of the state acquisition system 1 according to the fifth embodiment.

FIG. 20 is a diagram showing a functional configuration of the state acquisition system 1 according to a seventh embodiment.

FIG. 21 is a flowchart showing a processing procedure of the state acquisition system 1 according to the seventh embodiment.

FIG. 22 is a diagram in which values in a histogram of a dog in a state of excitement according to the seventh embodiment is represented with a broken line.

FIG. 23 is a histogram of a dog in a normal state in which breathing of the dog is stable according to the seventh embodiment.

FIG. 24 is a histogram of a dog in a normal state according to the seventh embodiment.

FIG. 25 is a histogram in a rest state of a dog according to the seventh embodiment.

FIG. 26 is a flowchart showing a processing procedure of the state acquisition system 1 according to an eighth embodiment.

FIG. 27 is a trajectory of a Poincare plot of a dog in a state of excitement according to the eighth embodiment.

FIG. 28 is a trajectory of a Poincare plot of a dog in a normal state in which breathing of the dog is stable according to the eighth embodiment.

FIG. 29 is a trajectory of a Poincare plot of a dog in a normal state according to the eighth embodiment.

FIG. 30 is a trajectory of a Poincare plot in a rest state of a dog according to the eighth embodiment.

FIG. 31 is a diagram showing a histogram or a pattern of a trajectory of a Poincare plot and an example in which matching degree thereof is represented by a numerical value according to a tenth embodiment.

FIG. 32 is a diagram showing a functional configuration of the state acquisition system 1 according to an eleventh embodiment.

FIG. 33 is a diagram showing a functional configuration of a first state acquisition system 1 according to a thirteenth embodiment.

FIG. 34 is a diagram showing a functional configuration of a second state acquisition system 1 according to the thirteenth embodiment.

FIG. 35 is a diagram showing a functional configuration of a third state acquisition system 1 according to the thirteenth embodiment.

FIG. 36 is a diagram showing a functional configuration of a fourth state acquisition system 1 according to the thirteenth embodiment.

FIG. 37 is a time series graph of products of standard deviations of a Poincare plot after axis conversion according to a fourteenth embodiment.

FIG. 38 is a diagram showing a mean value and variation of products of standard deviations of a Poincare plot after axis conversion according to the fourteenth embodiment.

FIG. 39 is a flowchart showing a first processing procedure of the state acquisition system 1 according to the fourteenth embodiment.

FIG. 40 is a flowchart showing a second processing procedure of the state acquisition system 1 according to the fourteenth embodiment.

FIG. 41 is a flowchart showing a processing procedure of the state acquisition system 1 according to the fifteenth embodiment.

FIG. 42 is a flowchart showing a processing procedure of the state acquisition system 1 according to a sixteenth embodiment.

FIG. 43 is a diagram for comparing mean values and variation of products of standard deviations of a Poincare plot after axis conversion at time A and time B according to the sixteenth embodiment.

FIG. 44 is a flowchart showing a processing procedure of the state acquisition system 1 according to a seventeenth embodiment.

FIG. 45 is a diagram for comparison after normalization of mean values and variation of products of standard deviations of a Poincare plot after axis conversion at time A and time B according to the seventeenth embodiment.

### DESCRIPTION OF EMBODIMENTS

Embodiments of the present disclosure will be described below with reference to drawings. In descriptions below, identical components are denoted by an identical reference sign. The identical components have an identical name and function. Therefore, descriptions thereof will not be repeated.

### First Embodiment

### Entire Configuration of State Acquisition System

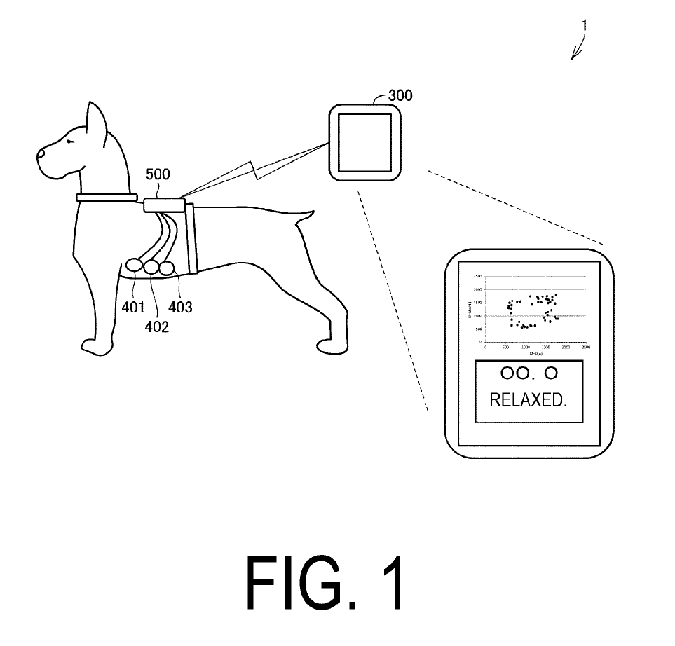

First, with reference to FIG. 1, a processing procedure of the state acquisition system 1 according to the present embodiment will be described. FIG. 1 is a diagram showing the entire configuration of the state acquisition system 1 according to the present embodiment. Note that, on behalf of an animal, a case will be described below in which a state of a dog having a respiratory sinus arrhythmia is determined.

The state acquisition system 1 according to the present embodiment mainly includes electrodes 401, 402, 403 that can be attached to the chest region of a dog, and is configured to acquire electrocardilgram, a signal processing apparatus 500 configured to process an electrocardiographic signal, and a communication terminal 300 that can communicate with the signal processing apparatus 500.

The electrodes 401, 402, 403 for acquiring the electrocardilgram are preferably attached to a position where the heart region is interposed on the chest region or the like, and may be attached to a hairless location such as a paw pad on both front feet (or a front foot and a hind foot), for example. Additionally, it is preferable that a dog be in a sheared state, gel or the like be adhered to the electrodes, or the electrodes have a protruding structure and have a configuration that contacts with skin even when the skin has hair. Alternatively, in a state where the skin has hair, a configuration is preferable in which the electrocardilgram is induced via a capacitive material in a non-contact manner. Accordingly, even in a case of an animal having surface skin covered with hair such as a dog, the electrocardilgram can be acquired. In the present embodiment, a configuration is adopted in which three electrodes 401, 402, 403 are used, but it is sufficient that at least two electrodes are used, or a configuration may be adopted in which more electrodes are used.

### Functional Configuration and Processing Procedure of State Acquisition System

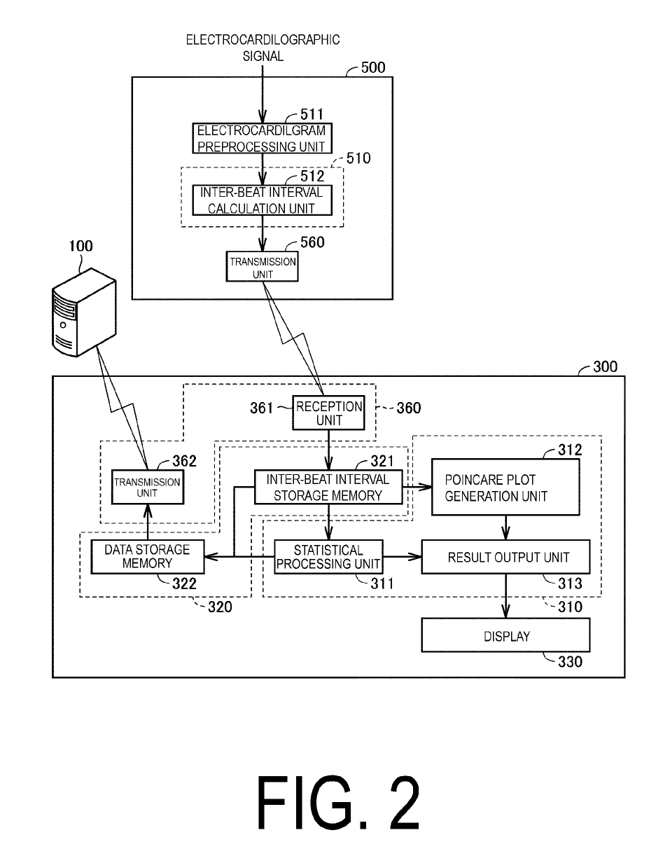

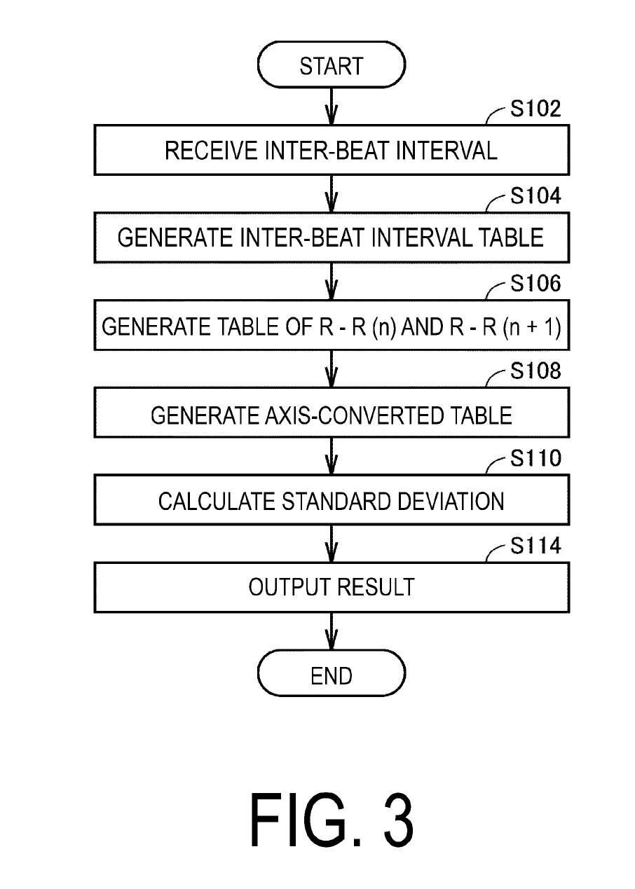

Next, with reference to FIG. 2 and FIG. 3, a functional configuration and a processing procedure of the state acquisition system 1 according to the present embodiment will be described. FIG. 2 is a diagram showing the functional configuration of the state acquisition system 1 according to the present embodiment. FIG. 3 is a flowchart showing the processing procedure of the state acquisition system 1 according to the present embodiment.

First, a configuration of the signal processing apparatus 500 in the state acquisition system 1 will be described. The signal processing apparatus 500 includes a electrocardilgram preprocessing unit 511, an inter-beat interval calculation unit 512, and a transmission unit 560.

The electrocardilgram preprocessing unit 511 includes a filter and an amplifier. The electrocardilgram preprocessing unit 511 converts an electrocardiographic signal transmitted from the electrodes 401, 402, 403 into pulsation data, and delivers the pulsation data to the inter-beat interval calculation unit 512.

More particularly, the electrocardilgram preprocessing unit 511 includes a filter device such as a high pass filter and a low pass filter, an amplification device formed of an operational amplifier and the like, an A/D conversion device configured to convert an analog signal of the electrocardilgram to a digital signal, and the like. Note that a configuration may be adopted in which the filter device, the amplification device, or the like is implemented by software. Moreover, the A/D conversion device preferably performs sampling in a period and with accuracy in which a difference between fluctuation amounts of the inter-beat intervals can be determined. That is, the A/D conversion frequency is preferably equal to 25 Hz or higher. For example, in the present embodiment, the electrocardiographic signal is sampled with 100 Hz. Increasing a sampling frequency enables the fluctuation amount of the inter-beat intervals to be precisely recognized.

The inter-beat interval calculation unit 512 is, for example, realized by a Central Processing Unit (CPU) 510 executing programs stored in a memory. The inter-beat interval calculation unit 512 sequentially calculates inter-beat intervals based on the pulsation data. More particularly, the inter-beat interval calculation unit 512 detects a peak signal (R wave) of the electrocardilgram by a method such as threshold value detection, and calculates a period (time) between the peak signals of the respective electrocardilgram. As a calculation method of the inter-beat interval, other than the above method, a method deriving a period by using an autocorrelation function or a method using a rectangular wave correlation trigger may be used.

In the present embodiment, the inter-beat interval calculation unit 512 sequentially executes calculation of the inter-beat intervals for sequentially inputted electrocardiographic signals. The inter-beat interval calculation unit 512 transmits the calculated inter-beat interval and the pulsation data themselves to the communication terminal 300 via the transmission unit 560. Note that the transmission unit 560 is realized by a communication interface including an antenna, a connector, or the like.

Next, a configuration of the communication terminal 300 will be described. The communication terminal 300 includes a reception unit 361, an inter-beat interval storage memory 321, a statistical processing unit 311, a Poincare plot generation unit 312, a result output unit 313, a display 330, a data storage memory 322, and a transmission unit 362.

First, the reception unit 361 and the transmission unit 362 are realized by a communication interface 360 including an antenna, a connector, or the like. The reception unit 361 receives data indicating the inter-beat interval from the signal processing apparatus 500 (step S102).

The inter-beat interval storage memory 321 is formed of various types of memories 320 and stores the data received from the signal processing apparatus 500. In the present embodiment, a CPU 310 sequentially stores the inter-beat intervals received via the communication interface 360 as an inter-beat interval table in the memory 320 (step S104). However, these data may be stored in the memory 320 of the communication terminal 300, or in other apparatuses that can be accessed from the communication terminal 300.

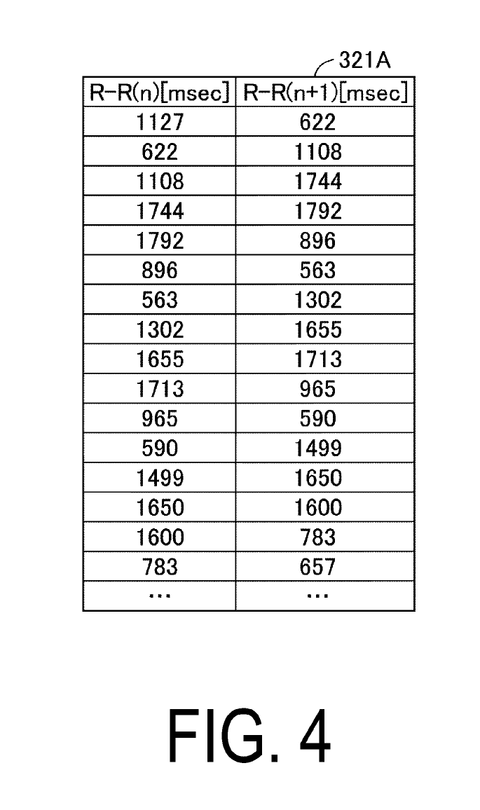

For example, the CPU 310 executes a program in the memory 320, so that the statistical processing unit 311, the Poincare plot generation unit 312, and the result output unit 313 are realized. The statistical processing unit 311 reads the inter-beat interval data from the inter-beat interval storage memory 321 at a fixed time unit, such as a minute, 10 minutes, or an hour, which is a time unit required for determining a state, for example, and generates a table 321A indicating correspondence relation between an inter-beat interval R-R (n) and the following inter-beat interval R-R (n+1), as shown in FIG. 4 (step S106). The inter-beat interval is calculated, for example, in the unit of milliseconds (msec), as shown in the figure.

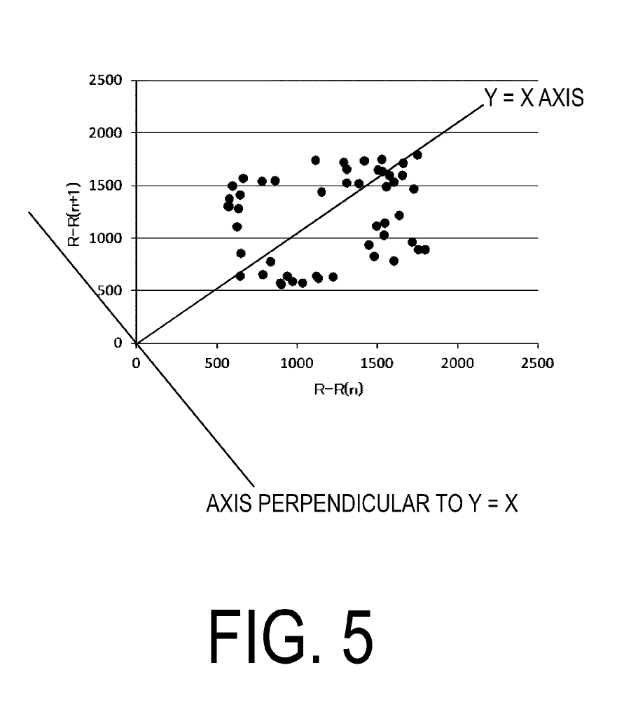

As shown in FIG. 5, the statistical processing unit 311 converts the table indicating correspondence relation between the inter-beat interval R-R (n) and the following inter-beat interval R-R (n+1) into axes in a Y=X direction and a direction perpendicular to the Y=X direction (step S108).

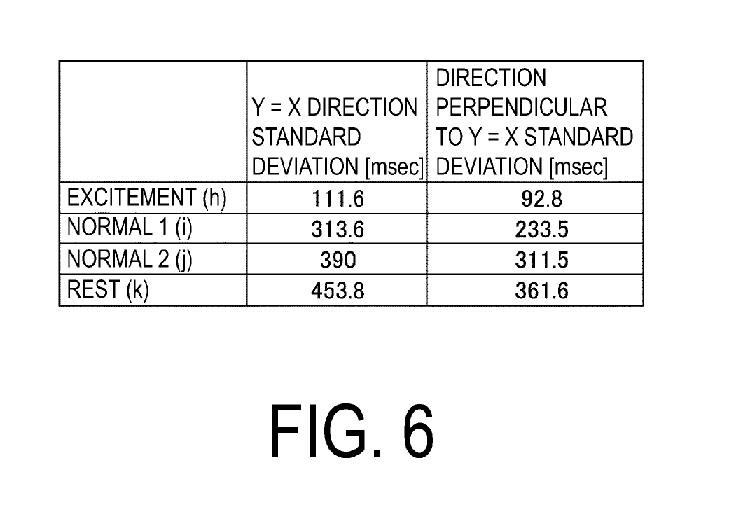

The statistical processing unit 311 calculates a standard deviation related to numerical series forming each axis after axis conversion (step S110). Note that the statistical processing unit 311 may calculate only a standard deviation related to a Y=X axis, only a standard deviation related to an axis perpendicular to the Y=X axis, or both the standard deviations. FIG. 6 is a table showing standards of standard deviations related to a Y=X axis and an axis perpendicular to the Y=X axis for each of psychological states or physical states of a dog.

Note that the statistical processing unit 311 may determine an axis in which a spread is maximum by using a method such as main component analysis to calculate standard deviations related to the axis and an axis perpendicular to the first-mentioned axis. Further, the statistical processing unit 311 may calculate standard deviations related to an X axis and a Y axis without performing the axis conversion. When directions in which spreads are large are an X axis direction and a Y direction, a variation state of the inter-beat intervals plotted by a Poincare plot can be evaluated by calculating the standard deviations related to the X axis and the Y axis without performing the axis conversion. In this case, a calculation amount can be reduced because the axis conversion is unnecessary.

The result output unit 313 allows an output device of itself or an external output device, such as the display 330 or a speaker, to display a standard deviation or output an audio message (step S114). Note that, to be specific, the result output unit 313 may output only a standard deviation related to a Y=X axis, only a standard deviation related to an axis perpendicular to the Y=X axis, both the standard deviations, only a larger one, or a smaller one.

By calculating a standard deviation, a variation state of the inter-beat intervals plotted by a Poincare plot with each of an inter-beat interval R-R (n) and the following inter-beat interval R-R (n+1) used as an axis can be evaluated.

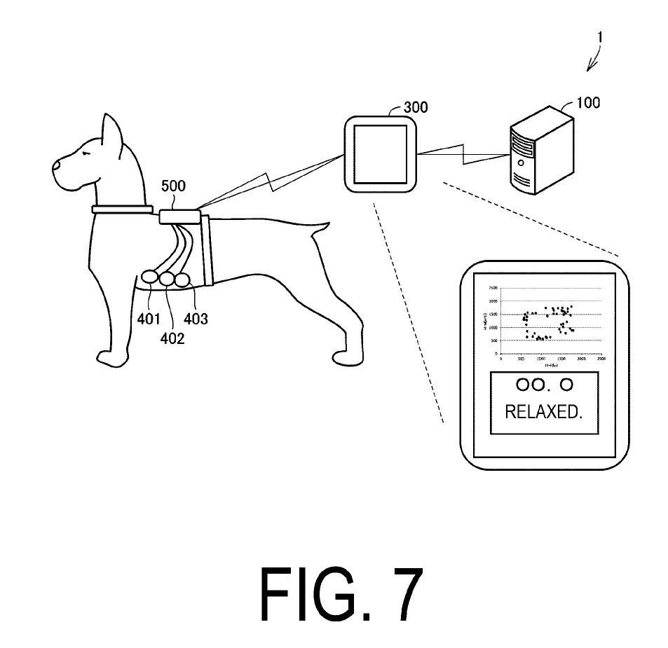

Note that, as shown in FIG. 7, the state acquisition system 1 according to the present embodiment may have a configuration including a server 100 with which the communication terminal 300 can communicate. In this case, the CPU 310 as the result output unit 313 stores a standard deviation, a relation table, or the like in the data storage memory 322, and uses the transmission unit 362 to perform transmission to the server 100 via the Internet. With this, the output result this time can be utilized to recognize a short-term or long-term stress state of an observation target.

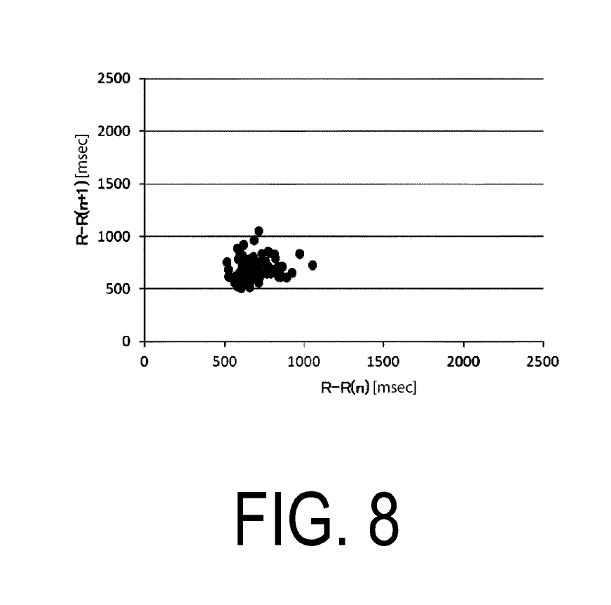

In the present embodiment, apart from the step S108, at the same time, the Poincare plot generation unit 312 acquires data of the inter-beat intervals R-R (n) and the following inter-beat intervals R-R (n+1) in a range that has been used in the standard deviation calculation from the correspondence relation table shown in FIG. 4 to generate the Poincare plot diagrams shown in FIG. 8 to FIG. 11.

Additionally, the result output unit 313 allows an output device of itself or an external output device such as a display to display the generated Poincare plot diagram. Note that the Poincare plot generation unit 312 may utilize the result in the step S108 to generate and output the Poincare plot diagram after the axis conversion.

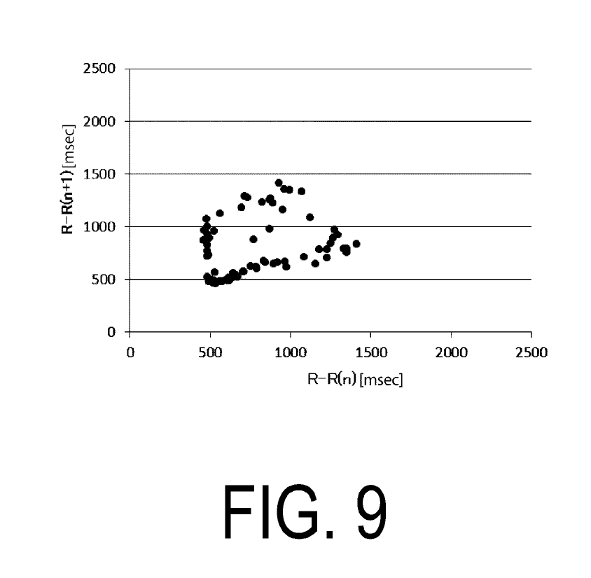

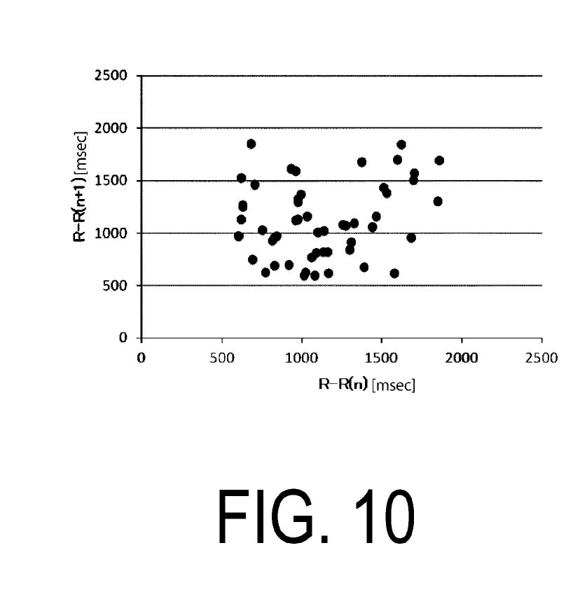

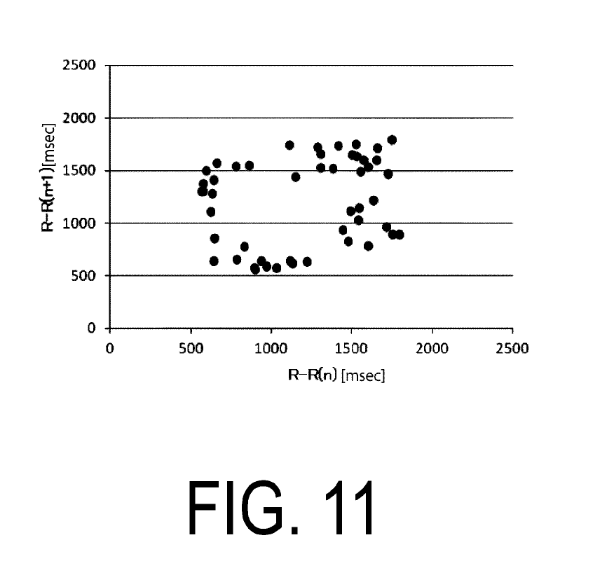

Hereinafter, the Poincare plot diagram will be described. FIG. 8 is a Poincare plot diagram of a dog in a state of excitement according to the present embodiment. FIG. 9 is a Poincare plot diagram of a dog in a normal state in which breathing of the dog is stable according to the present embodiment. FIG. 10 is a Poincare plot diagram of a dog in a normal state according to the present embodiment. FIG. 11 is a Poincare plot diagram in a rest state of a dog according to the present embodiment.

First, in a case of an animal having a respiratory sinus arrhythmia, such as a dog, in an excited state as in FIG. 8, a heart rate increases (an inter-beat interval becomes shorter), fluctuation of the inter-beat interval becomes smaller, and plot points are in a state of gathering at a certain place.

In addition, in a normal state in which breathing is stable as in FIG. 9, a heart rate is not so low compared with that in a rest state (a spread of plot points is not so large compared with that in the rest state), but a region where the number of the plot points is small (a blank hole) exists in a center portion of distribution of the plot points. A reason of such a shape is that pulsation fluctuation periodically changes because a heart rate of a dog is largely influenced by breathing (respiratory sinus arrhythmia). Accordingly, although the pulsation is not slow in a relaxed state, since the breathing is stably performed, the blank exists.

Additionally, in a normal state as in FIG. 10, although pulsation fluctuation occurs and variation becomes larger (plot points spread), the plot points are in a scattered state.

Further, in a rest state as in FIG. 11, an inter-beat interval becomes larger because a dog is in a relaxed state, and since a respiratory sinus arrhythmia largely influences the plot points, a spread of plot points becomes larger, and a shape of the plot points becomes close to a circle, a rectangle, or a triangle. In any shape among the above-described shapes, in the rest state, distribution of the plot points of a Poincare plot becomes a shape having a blank part in a center portion.

Therefore, in the present embodiment, the degree of the distribution spread of the plot points of the Poincare plot, the size, the shape, and whether the number of the plot points in the center portion is large or small, can be indirectly estimated based on a calculation result, and as a result, a psychological state or a physical state of an animal can be estimated.

### Second Embodiment

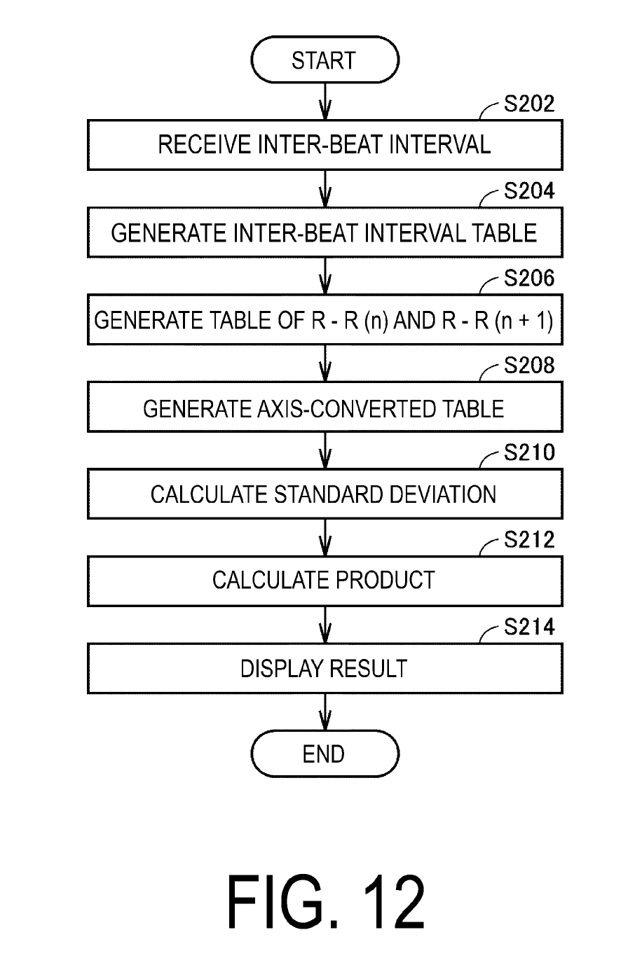

In the first embodiment, the communication terminal 300 outputs the standard deviation along the Y=X axis or the standard deviation along the axis perpendicular to the Y=X axis, of the Poincare plot. However, in the present embodiment, a product of these two standard deviations is calculated. With reference to FIG. 12, a processing procedure of the state acquisition system 1 according to the present embodiment will be described below.

FIG. 12 is a flowchart showing the processing procedure of the state acquisition system 1 according to the present embodiment. Since a step S202 to a step S208 are identical to the step S102 to the step 108 in the first embodiment, the description will not be repeated hereinafter.

The CPU 310 as the statistical processing unit 311 calculates a standard deviation related to each axis after the axis conversion (step S210). Note that the statistical processing unit 311 may determine an axis in which a spread is maximum to calculate standard deviations related to the axis and an axis perpendicular to the first-mentioned axis.

Additionally, the statistical processing unit 311 calculates the product of these two standard deviations (step S212).

The result output unit 313 allows, for example, an output device of itself or an external output device, such as a display or a speaker, to display a product of standard deviations or output an audio message (step S214). More particularly, the result output unit 313 may output a standard deviation related to the Y=X axis, a standard deviation related to the axis perpendicular to the Y=X axis, and a product of both the standard deviations.

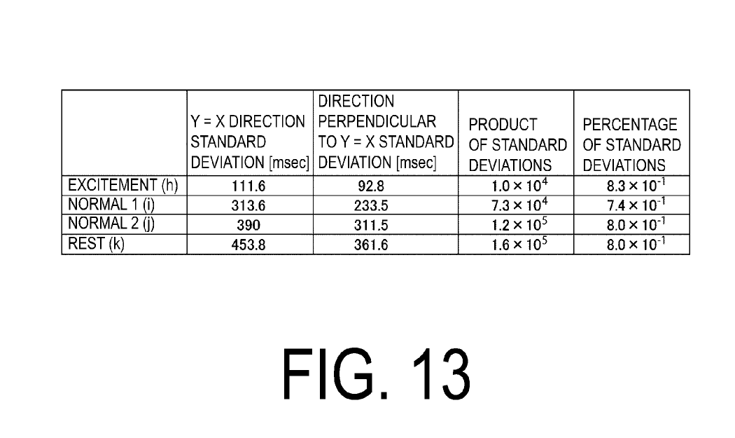

FIG. 13 is a table showing standards of standard deviations related to the Y=X axis and the axis perpendicular to the Y=X axis, a product of the standard deviations, and a ratio of the standard deviations for each of psychological states or physical states of a dog.

By calculating a product of standard deviations, as for a distribution spread of the inter-beat intervals plotted by a Poincare plot with each of an inter-beat interval R-R (n) and the following inter-beat interval R-R (n+1) used as an axis, a variation state, that is, a size and a shape of the spread, whether the plot points are uniformly distributed, whether a blank exists in a center portion, or the like can be evaluated. Moreover, even in a state where an aspect ratio is identical but only the size changes, or even in a case where a distribution spread area is identical but the variation state is different, the variation state can be efficiently evaluated.

In this case, the result output unit 313 stores a standard deviation, a product of the standard deviations, a correspondence relation table, or the like in the data storage memory 322, and uses the transmission unit 362 to perform transmission to the server 100 via the Internet. With this, the output result this time can be utilized to recognize a short-term or long-term stress state of an observation target.

Also in the present embodiment, the CPU 310 as the Poincare plot generation unit 312 may generate Poincare plot diagrams as shown in FIG. 8 to FIG. 11, allow the generated Poincare plot diagram to be displayed by an output device such as a display, and transmit the generated Poincare plot diagram to the server 100.

Note that, in the present embodiment, although the statistical processing unit 311 calculates a product of standard deviations related to two axes, a product of standard deviations related to three or more axes may be calculated.

Therefore, in the present embodiment, the degree of the distribution spread of the plot points of the Poincare plot, the size, the shape, and whether the number of the plot points in the center portion is large or small, can be indirectly estimated based on a calculation result, and as a result, a psychological state or a physical state of an animal can be estimated.

### Third Embodiment

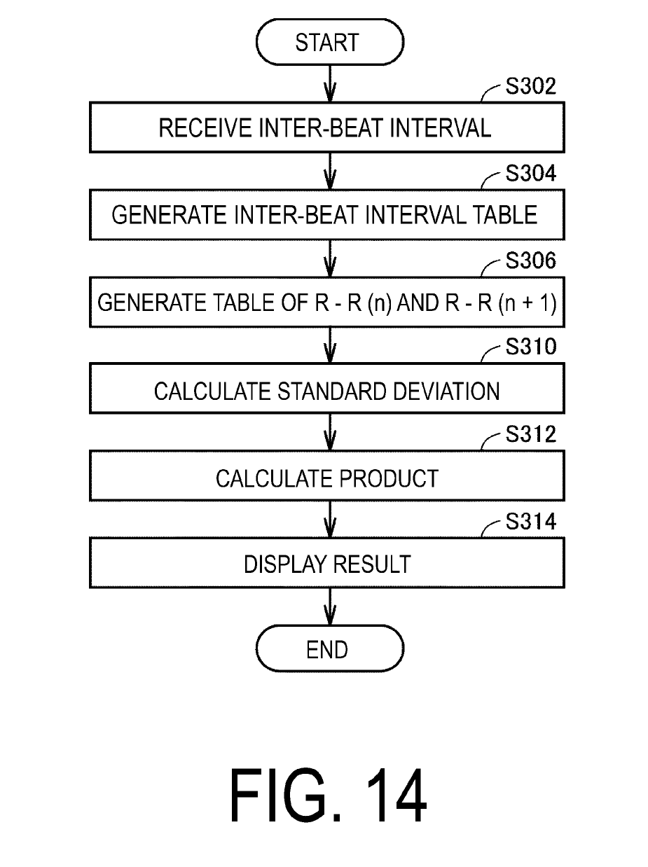

In the first embodiment, the communication terminal 300 outputs the standard deviation related to the Y=X axis or the standard deviation related to the axis perpendicular to the Y=X axis, of the Poincare plot. However, in the present embodiment, a standard deviation related to an X axis or a Y axis of a Poincare plot before axis conversion or a product of standard deviations is calculated. With reference to FIG. 14, a processing procedure of the state acquisition system 1 according to the present embodiment will be described below.

FIG. 14 is a flowchart showing the processing procedure of the state acquisition system 1 according to the present embodiment. Since a step S302 to a step S304 are identical to the step S102 to the step 104 in the first embodiment, the description will not be repeated hereinafter.

The statistical processing unit 311 reads the inter-beat interval data from the inter-beat interval storage memory 321 at a fixed time unit, such as a minute, 10 minutes, or an hour, which is a time unit required for determining a state, for example, and generates a relation table between an inter-beat interval R-R (n) and the following inter-beat interval R-R (n+1) (step S306).

The statistical processing unit 311 calculates a standard deviation related to the X axis and a standard deviation related to the Y axis (step S310).

Additionally, the statistical processing unit 311 calculates the product of these two standard deviations (step S312).

The CPU 310 as the result output unit 313 allows, for example, an output device, such as a display or a speaker, to display a product of standard deviations or output an audio message (step S314). More particularly, the result output unit 313 may output a standard deviation related to the X axis, a standard deviation related to the Y axis, and a product of both the standard deviations.

By calculating a product of standard deviations, a variation state of the inter-beat intervals plotted by a Poincare plot with each of an inter-beat interval R-R (n) and the following inter-beat interval R-R (n+1) used as an axis can be evaluated.

Note that, also in the present embodiment, the CPU 310 as the result output unit 313 stores a standard deviation, a product of the standard deviations, a correspondence relation table, or the like in the data storage memory 322, and uses the transmission unit 362 to perform transmission to the server 100 via the Internet. With this, the output result this time can be utilized to recognize a short-term or long-term stress state of an observation target.

Also in the present embodiment, the CPU 310 as the Poincare plot generation unit 312 may generate Poincare plot diagrams as shown in FIG. 8 to FIG. 11, allow the generated Poincare plot diagram to be displayed by an output device such as a display, and transmit the generated Poincare plot diagram to the server 100.

Note that, the excited state, a first normal state (a normal and stable breathing state), a second normal state, and the rest state in the first embodiment to the third embodiment can be used as an index indicating a stress state. These states correlate with LF/HF analysis of the inter-beat interval. In the state in FIG. 8, an LF/HF value is large, and as the state transits to FIG. 9, FIG. 10, FIG. 11 in order, the LF/HF value becomes smaller. This means a relaxed state. Accordingly, transition of a stress state of a dog can be recognized. However, LF/HF has a limit in a range that can be distinguished, and thus the state can be distinguished in detail by using the Poincare plot. In addition, using the Poincare plot has an advantage that a calculation amount decreases, because frequency analysis such as LF/HF analysis is unnecessary.

Therefore, in the present embodiment, the degree of the distribution spread of the Poincare plot, the size, the shape, and whether the number of the plot points in the center portion is large or small, can be estimated based on a calculation result, and as a result, a psychological state or a physical state of an animal can be estimated.

### Fourth Embodiment

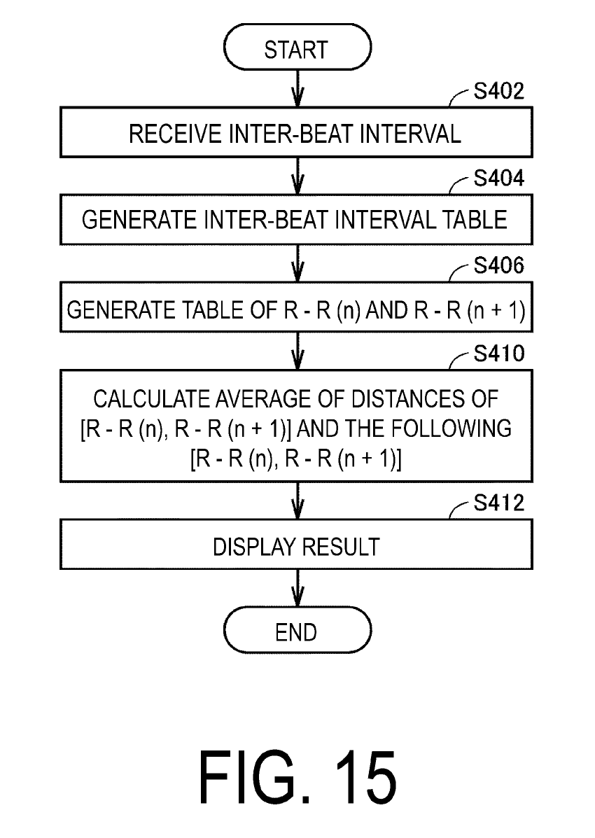

In the first embodiment to the third embodiment, the communication terminal 300 outputs the standard deviation or the product of the standard deviations of the Poincare plot. However, in the present embodiment, an average of distances between two sequential plots of the Poincare plot is calculated. With reference to FIG. 15, a processing procedure of the state acquisition system 1 according to the present embodiment will be described below.

FIG. 15 is a flowchart showing the processing procedure of the state acquisition system 1 according to the present embodiment. Since a step S402 to a step S406 are identical to the step S102 to the step 106 in the first embodiment, the description will not be repeated hereinafter.

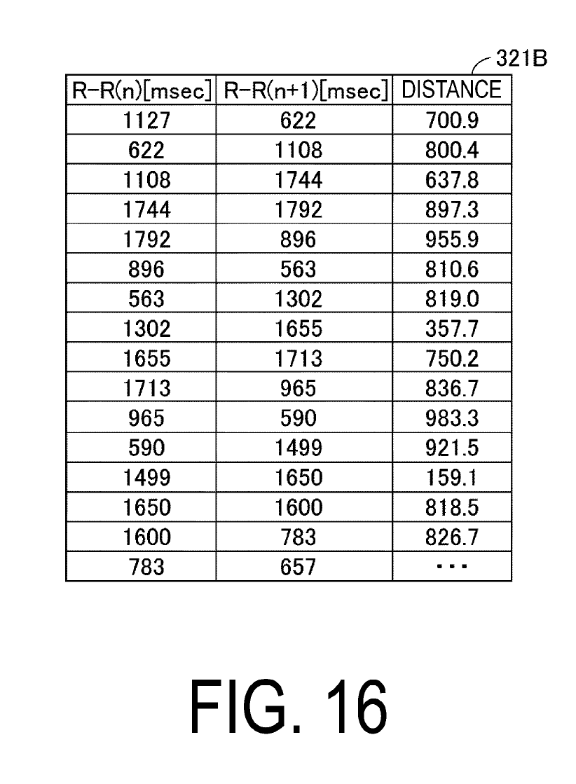

The CPU 310 as the statistical processing unit 311 calculates distances from [R-R (n), R-R (n+1)] to the following [R-R (n), R-R (n+1)] in a certain period, and stores the distance table 321B as shown in FIG. 16 in the memory 320. The CPU 310 calculates an average of the calculated distances (step S410). Note that the statistical processing unit 311 may determine an axis in which a spread is maximum and calculate the average of the distances after conversion to the determined axis.

The CPU 310 as the result output unit 313 allows, for example, an output device, such as a display or a speaker, to display the average of the distances or output an audio message (step S412).

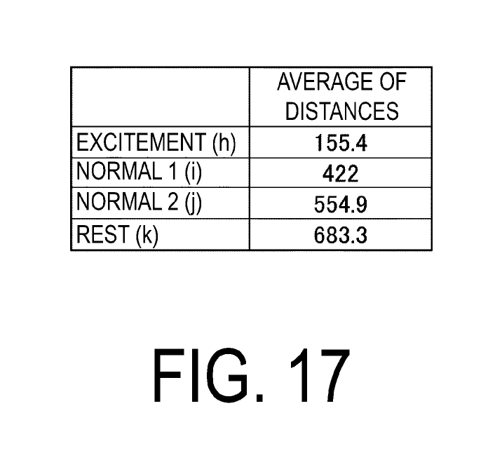

FIG. 17 is a table showing a standard of an average of distances for each of psychological states or physical states of a dog according to the present embodiment.

Since the distances between the respective points are different due to the distribution state of the Poincare plot, calculating the average can allow the shape of the Poincare plot to be reflected. Therefore, in the present embodiment, the degree of the distribution spread of the Poincare plot, the size, the shape, and whether the number of the plot points in the center portion is large or small, can be estimated based on a calculation result, and as a result, a psychological state or a physical state of an animal can be estimated.

### Fifth Embodiment

In the first embodiment to the fourth embodiment, the communication terminal 300 outputs the Poincare plot itself, the standard deviation, the product of the standard deviations, the average of the distances, or the like. However, in the present embodiment, based on these numerical values, a state of an animal is also determined.

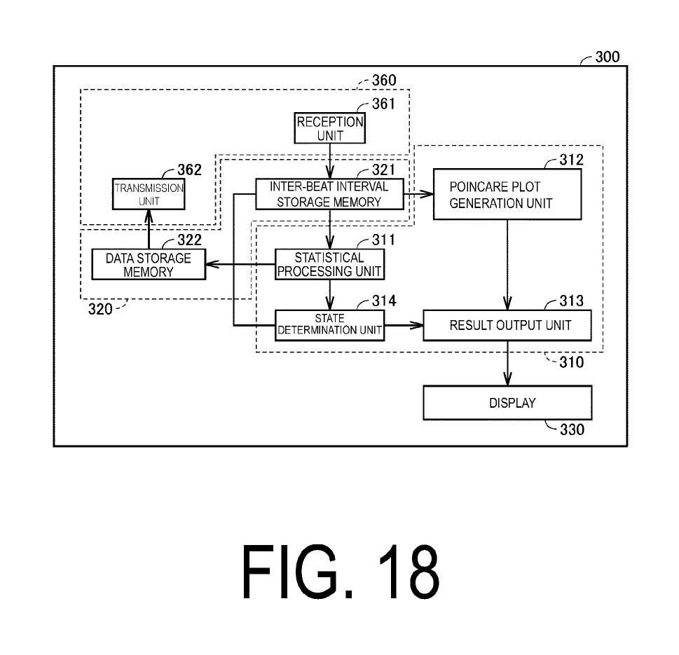

First, with reference to FIG. 18, a functional configuration of the state acquisition system 1 according to the present embodiment will be described. FIG. 18 is a diagram showing the functional configuration of the state acquisition system 1 according to the present embodiment.

The communication terminal 300 includes a state determination unit 314, compared with the communication terminal 300 according to the first embodiment to the fourth embodiment. For example, the CPU 310 executes a program in the memory 320, so that the state determination unit 314 is realized.

The memory 320 in the communication terminal 300 stores a numerical value range and model feature data for each state of an animal. For example, the memory 320 stores a numerical value range or a threshold value of the standard deviation for each state. Alternatively, the memory 320 stores a numerical value range or a threshold value of the product of the standard deviations for each state. Alternatively, the memory stores a numerical value range or a threshold value of the average of the distances for each state. Alternatively, the memory 320 stores a model shape of the Poincare plot of each state.

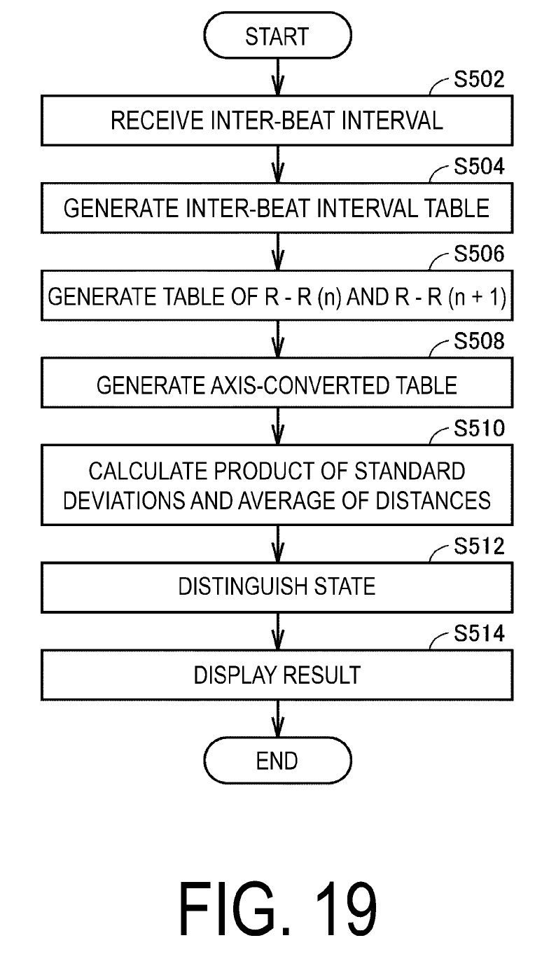

With reference to FIG. 19, a processing procedure of the state acquisition system 1 according to the present embodiment will be described below. FIG. 19 is a flowchart showing the processing procedure of the state acquisition system 1 according to the present embodiment. Since a step S502 to a step S508 are identical to the step S102 to the step 108 in the first embodiment, the description will not be repeated hereinafter.

The statistical processing unit 311 calculates the standard deviation, the product of the standard deviations, the average of the distances, or the like (step S510). Note that, here, the Poincare plot generation unit 312 may generate the Poincare plot.

Next, the CPU 310 as the state determination unit 314 determines the numerical value range corresponding to the state within which the standard deviation from the statistical processing unit 311 falls (step S512). Alternatively, the state determination unit 314 determines the numerical value range corresponding to the state within which the product of the standard deviations from the statistical processing unit 311 falls. Alternatively, the state determination unit 314 determines the numerical value range corresponding to the state within which the average of the distances from the statistical processing unit 311 falls. The state determination unit 314 determines the model shape of the Poincare plot corresponding to the state to which the Poincare plot shape from the statistical processing unit 311 or the Poincare plot generation unit 312 is similar.

For example, the memory 320 as the data storage memory 322 stores data, smaller than 5.0×10⁴ in the excited state, equal to or larger than 5.0×10⁴ and smaller than 1.0×10⁵ in the first normal state, equal to or larger than 1.0×10⁵ and smaller than 1.4×10⁵ in the second normal state, and equal to or larger than 1.4×10⁵ in the rest state, as the numerical value ranges of the product of the standard deviations. Moreover, the state determination unit 314 determines the state based on the data.

Alternatively, the memory 320 as the data storage memory 322 stores data, smaller than 4.0×10² in the excited state, equal to or larger than 4.0×10² and smaller than 5.0×10² in the first normal state, equal to or larger than 5.0×10² and smaller than 6.0×10² in the second normal state, and equal to or larger than 6.0×10² in the rest state, as the numerical value ranges of the average of the distances. Additionally, the state determination unit 314 determines the numerical value range corresponding to the state within which the calculation result from the statistical processing unit 311 falls.

The CPU 310 as the result output unit 313 allows, for example, an output device, such as a display or a speaker, to display the standard deviation, the product of the standard deviations, the average of the distances, the Poincare plot graph itself, and the state of the animal as the determination result or output an audio message (step S514).

Note that the result output unit 313 stores the calculation result, the determination result, or the like in the data storage memory 322, and uses the transmission unit 362 to transmit the calculation result, the determination result, or the like to the server 100 via the Internet. With this, the output result this time can be utilized to recognize a short-term or long-term stress state of an observation target.

Therefore, in the present embodiment, the degree of the distribution spread of the Poincare plot, the size, the shape, and whether the number of the plot points in the center portion is large or small, can be estimated based on the calculation result, and as a result, a psychological state or a physical state of an animal can be estimated. That is, as for an animal, due to a psychological state (excitement, anger, delight, sadness, fun, stress state, stress-free state), a variation state of inter-beat intervals changes. In addition, also as for change of a physical state (due to exercise, body motion, sleep, rest, disease, pain), a variation state of inter-beat intervals changes. The change of these state can be estimated. Recognizing the state change enables mutual understanding or communication support between an animal and a human, training, exercise load adjustment, or the like. Further, detecting the change in the psychological state or the physical state due to disease can be useful for treatment or disease observation.

### Sixth Embodiment

Note that, in FIG. 19, the statistical processing unit 311 may acquire a plurality of indexes among the standard deviation, the product of the standard deviations, the average of the distances, the Poincare plot graph, or the like (step S510). Moreover, the state determination unit 314 may determine the state of the animal, based on the plurality of indexes (step S512). In this case, the state determination unit 314 may determine the state based on the index with higher matching degree, or may determine the most unstable state among a plurality of the determination results as the determination result.

### Seventh Embodiment

In the first embodiment to the sixth embodiment, the Poincare plot is mainly used for determining the state of the animal. In the present embodiment, the histogram of the inter-beat intervals is used for determining the state of the animal.

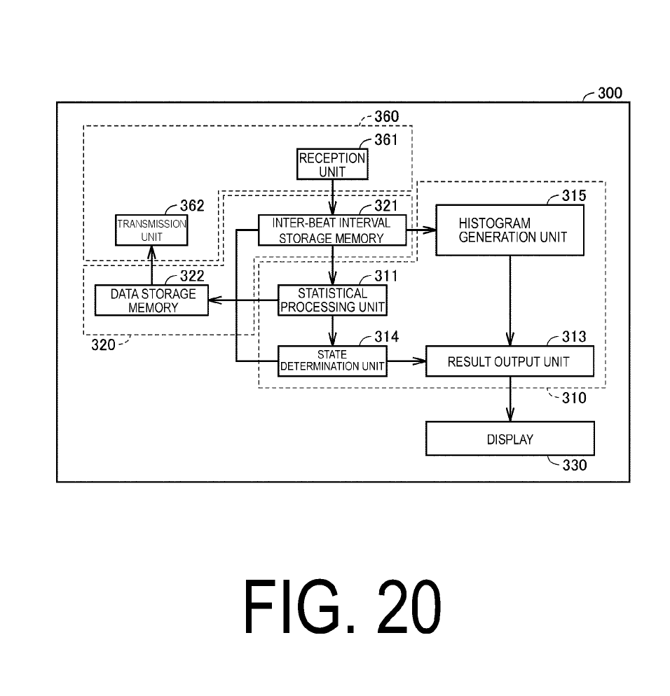

First, with reference to FIG. 20, a functional configuration of the state acquisition system 1 according to the present embodiment will be described. FIG. 20 is a diagram showing the functional configuration of the state acquisition system 1 according to the present embodiment.

The communication terminal 300 includes a histogram generation unit 315, instead of the Poincare plot generation unit 312, compared with the communication terminal 300 according to the fifth embodiment. For example, the CPU 310 executes a program in the memory 320, so that the histogram generation unit 315 is realized.

The memory 320 in the communication terminal 300 stores histogram model data or the like for each state of the animal.

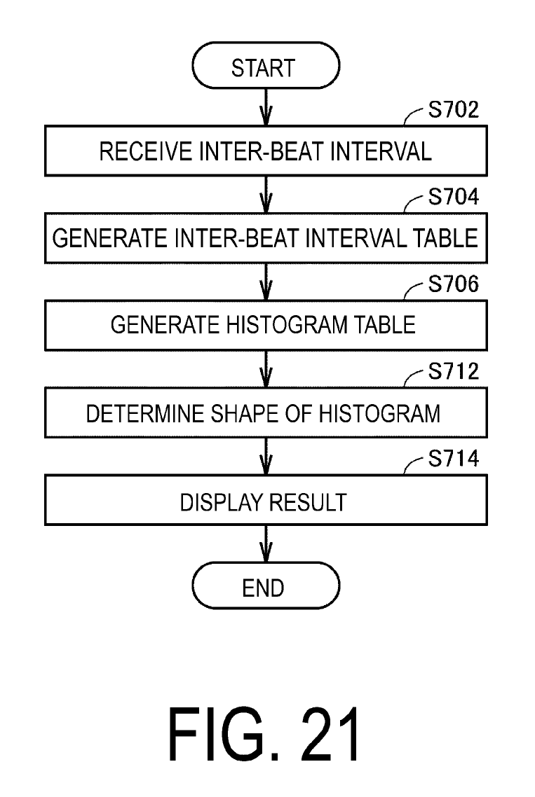

With reference to FIG. 21, a processing procedure of the state acquisition system 1 according to the present embodiment will be described below. FIG. 21 is a flowchart showing the processing procedure of the state acquisition system 1 according to the present embodiment. Since a step S702 to a step S704 are identical to the step S102 to the step 104 in the first embodiment, the description will not be repeated hereinafter.

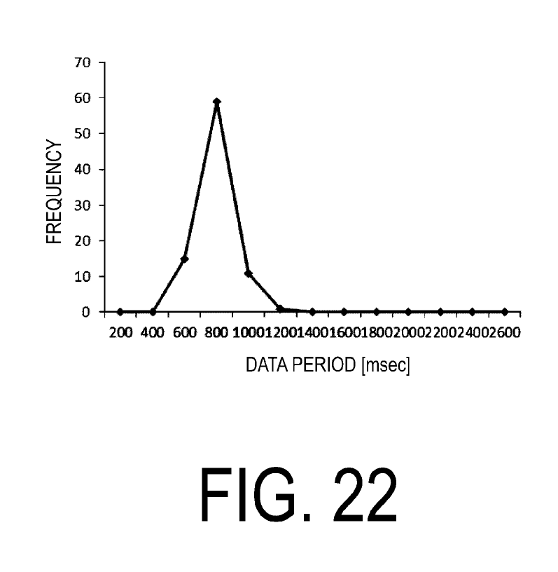

The CPU 310 as the statistical processing unit 311 reads the inter-beat interval data from the inter-beat interval storage memory 321 at a fixed time unit, such as a minute, 10 minutes, or an hour, which is a time unit required for determining a state, for example, and generates a histogram indicating relation between the inter-beat interval and its frequency, as shown in FIG. 22 to FIG. 25 (step S706).

Next, the CPU 310 as the state determination unit 314 determines the state corresponding to the histogram model shape to which a shape of the histogram from the histogram generation unit 315 is similar (step S712).

The CPU 310 as the result output unit 313 allows, for example, an output device, such as a display or a speaker, to display the histogram itself and/or the state of the animal as the determination result, or output an audio message (step S714).

Note that the result output unit 313 stores the calculation result, the determination result, or the like in the data storage memory 322, and uses the transmission unit 362 to transmit the calculation result, the determination result, or the like to the server 100 via the Internet. With this, the output result this time can be utilized to recognize a short-term or long-term stress state of an observation target.

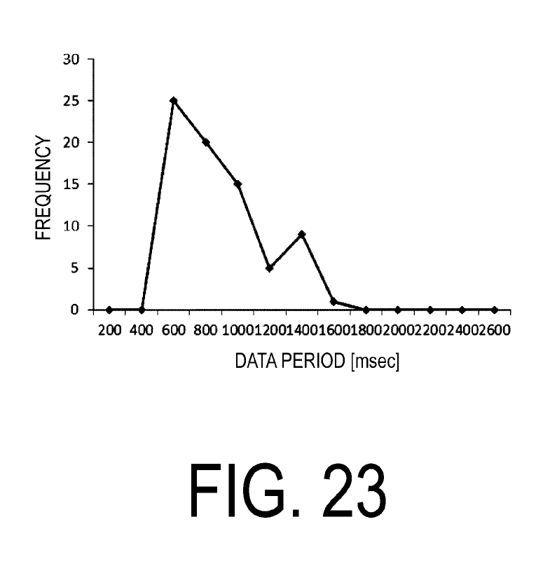

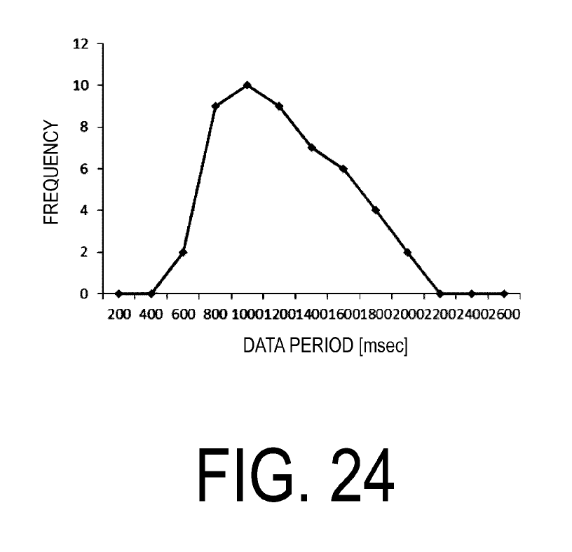

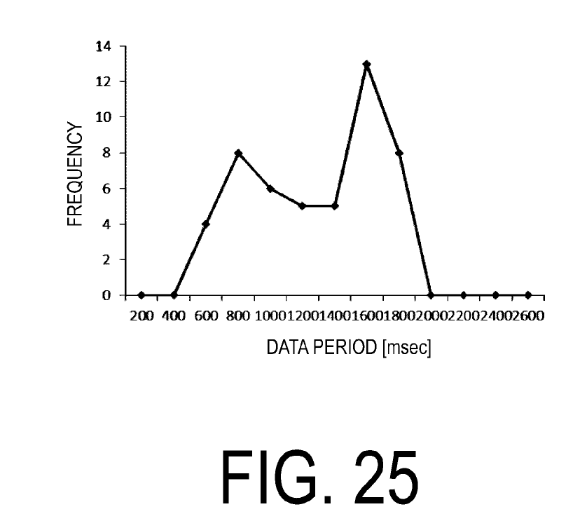

For example, in a case of a dog, in the excited state, as shown in FIG. 22, one peak with a narrow half value width appears. Further, in the first normal state, as shown in FIG. 23, two peaks with relatively narrow half value widths appear, reflecting the fact that the blank is in the center portion in the Poincare plot. Additionally, as shown in FIG. 24, in the second normal state, although the number of peaks is one, a peak with a wide half value width appears. As shown in FIG. 25, in the rest state, two peaks appear reflecting the fact that the blank is in the center portion in the Poincare plot.

The state determination unit 314 distinguishes a model to which the shape of this histogram is similar, specifies the corresponding state, and thus determines the excited state, the first normal state, the second normal state, the rest state, or the like. For example, the state determination unit 314 detects the number of peaks by peak detection, and, by obtaining the half value width (spread of a skirt of the peak), determines spread degree of the distribution by using the threshold value. In this case, the memory 320 stores the numerical value range indicating the distribution spread of the histogram for each state.

Specifically, the state determination unit 314 determines the excited state in a case of one peak with the narrow half value width. The state determination unit 314 determines the second normal state in a case of one peak with the wide half value width. The state determination unit 314 determines the first normal state in a case of two peaks with the narrow half value widths. The state determination unit 314 determines the rest state in a case of two peaks with the wide half value widths.

Note that since these threshold values and numerical value ranges are possibly different depending on a type or an age of a dog, the settings are preferably changed in accordance with these conditions. For example, a configuration may be provided in which a user inputs a type, a gender, and an age of a dog, and then threshold values are set in accordance with the type, the gender and the age of the dog.

With this, the histogram is generated by counting the number of pulsations for each of lengths of the inter-beat intervals, and the state can be distinguished by using the distribution of the histogram. The calculation amount can be reduced before the state is distinguished. Additionally, in the present embodiment, the degree of the distribution spread of the Poincare plot, the size, the shape, and whether the number of the plot points in the center portion is large or small, can be estimated based on the calculation result, and as a result, a psychological state or a physical state of an animal can be estimated.

### Eighth Embodiment

Alternatively, by using the trajectory of the Poincare plot, the state of the animal may be determined. In the present embodiment, the CPU 310 as the Poincare plot generation unit 312 in FIG. 18 acquires data of the inter-beat intervals R-R (n) and the following inter-beat intervals R-R (n+1) in a range that has been used in the standard deviation calculation from the correspondence relation table to generate the Poincare plot diagrams shown in FIG. 8 to FIG. 11. More particularly, in the present embodiment, the Poincare plot generation unit 312 not only performs plotting but also acquires, recognizes and draws the trajectory from a plot to the following plot.

Additionally, the memory 320 in the communication terminal 300 stores trajectory model data of the Poincare plot or the like for each state of the animal.

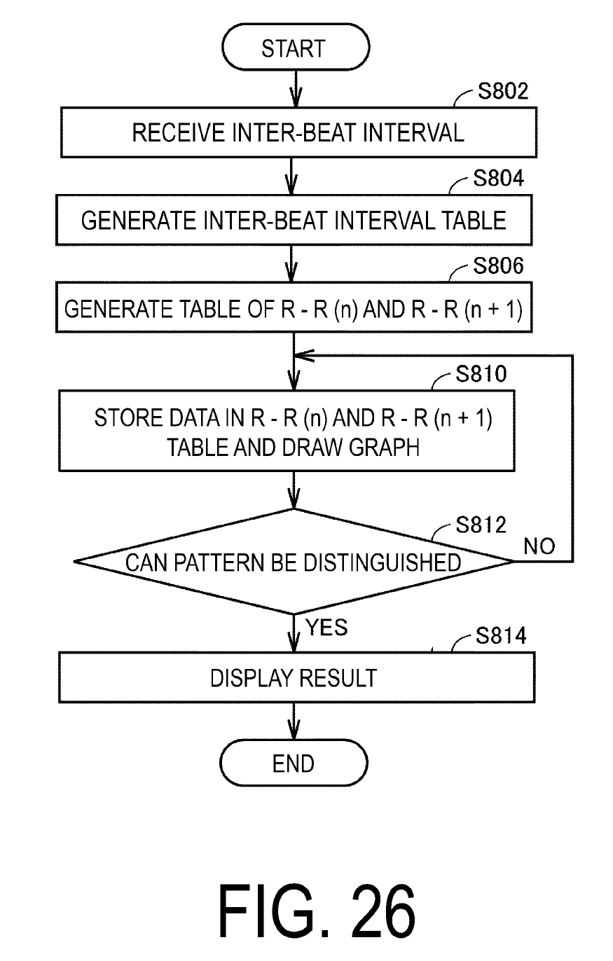

With reference to FIG. 26, a processing procedure of the state acquisition system 1 according to the present embodiment will be described below. FIG. 26 is a flowchart showing the processing procedure of the state acquisition system 1 according to the present embodiment. Note that since a step S802 to a step S806 are identical to the step S102 to the step 106 in the first embodiment, the description will not be repeated hereinafter.

As shown in FIG. 5, the CPU 310 as the statistical processing unit 311 draws the Poincare plot graph including the trajectory between the plots from the table 321A indicating correspondence relation between the inter-beat interval R-R (n) and the following inter-beat interval R-R (n+1) (step S810).

Next, the state determination unit 314 refers to the memory 320, and determines whether there is a trajectory model similar to the drawn trajectory of the Poincare plot or not by technology such as pattern recognition (step S812). In a case where the drawn trajectory of the Poincare plot is not similar to any trajectory model (in a case of NO in the step S812), the CPU 310 reads correspondence relation of the inter-beat intervals in another timing, and repeats the processing from the step S810 again.

In a case where there is a trajectory model similar to the drawn trajectory of the Poincare plot (in a case of YES in the step S812), the CPU 310 as the result output unit 313 allows, for example, an output device, such as a display or a speaker, to display the trajectory graph of the Poincare plot itself and/or the state of the animal as the determination result, or output an audio message (step S814).

Note that the CPU 310 as the result output unit 313 stores the calculation result, the determination result, or the like in the data storage memory 322, and uses the transmission unit 362 to transmit the calculation result, the determination result, or the like to the server 100 via the Internet. With this, the output result this time can be utilized to recognize a short-term or long-term stress state of an observation target.

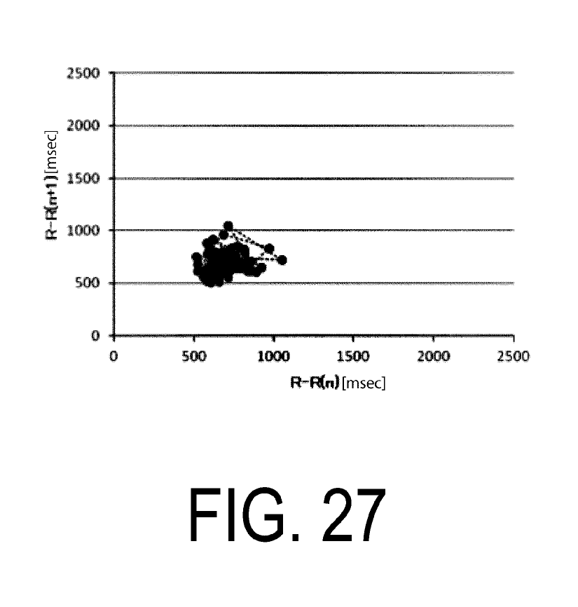

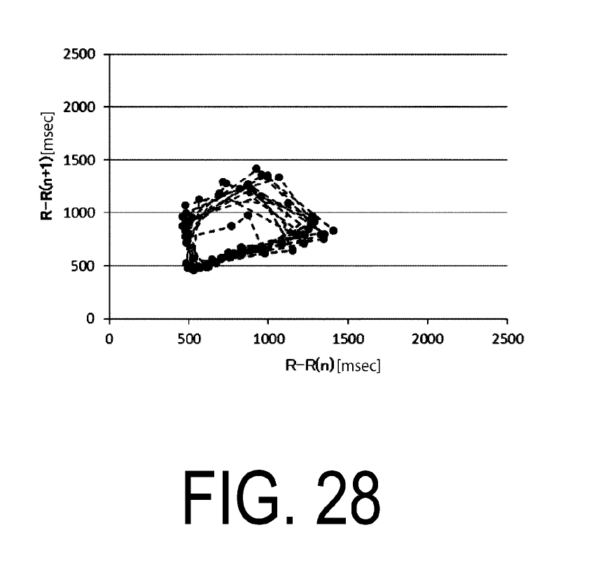

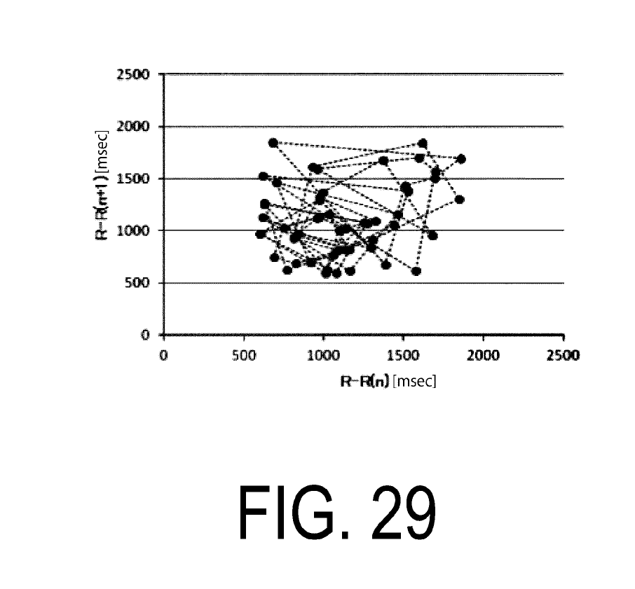

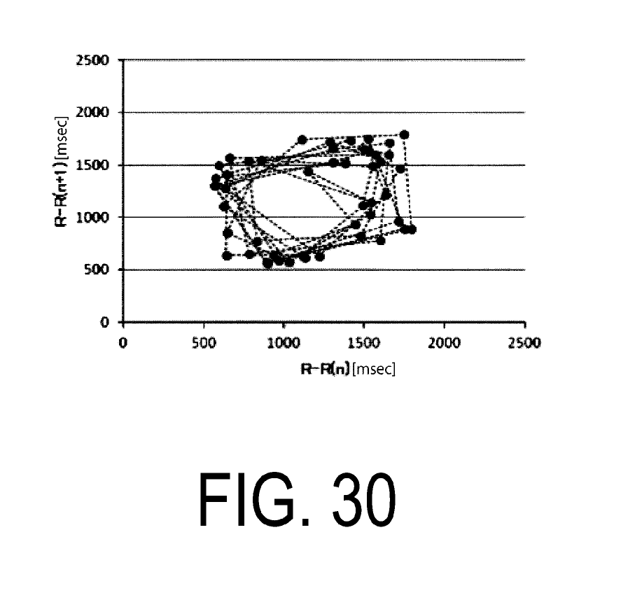

For example, in a case of a dog, the trajectory shown in FIG. 27 is drawn in the excited state, the trajectory shown in FIG. 28 is drawn in the first normal state, the trajectory shown in FIG. 29 is drawn in the second normal state, and the trajectory shown in FIG. 30 is drawn in the rest state.

Therefore, in the present embodiment, the degree of the distribution spread of the Poincare plot, the size, the shape, and whether the number of the plot points in the center portion is large or small, can be estimated based on the calculation result, and as a result, a psychological state or a physical state of an animal can be estimated.

### Ninth Embodiment

In order to determine the rest state, it is sufficient that the state acquisition system 1 can determine whether there is an area where the number of the plots are small in the center portion of the plots in the graph of the Poincare plot or not, and as the determination method, a plurality of methods among the first embodiment to the eighth embodiment may be used, or a method different from the methods among the first embodiment to the eighth embodiment may be used. In other words, the state acquisition system 1 may determine the state of the animal in accordance with an amount or a percentage of the plots in the center portion among the entire plots in the graph of the Poincare plot.

For example, the state determination unit 314 may determine the rest state, in a case where the number of plots is only smaller than a first prescribed percentage to the total number of plots, within a distance from an average of all plots to the standard deviation multiplied by prescribed times with relation to each of two axes in the graph of the Poincare plot. Additionally, the state determination unit 314 may determine the normal state, in a case where the number of plots is only equal to or larger than the first prescribed percentage and smaller than a second prescribed percentage to the total number of plots, within the distance from the average of all plots to the standard deviation multiplied by prescribed times with relation to each of two axes in the graph of the Poincare plot. Additionally, the state determination unit 314 may determine the excited state, in a case where the number of plots is equal to or larger than the second prescribed percentage to the total number of plots, within the distance from the average of all plots to the standard deviation multiplied by prescribed times with relation to each of two axes in the graph of the Poincare plot.

Specifically, the state determination unit 314 may determine the rest state, in a case where the number of plots is only smaller than 10 percent to the total number of plots, within the distance from the average of all plots to the standard deviation multiplied by 0.5 with relation to each of two axes in the graph of the Poincare plot. In addition, the state determination unit 314 may determine the normal state, in a case where the number of plots is only equal to or larger than 10 percent and smaller than 30 percent to the total number of plots, within the distance from the average of all plots to the standard deviation multiplied by 0.5 with relation to each of two axes in the graph of the Poincare plot. In addition, the state determination unit 314 may determine the excited state, in a case where the number of plots is equal to or larger than 30 percent to the total number of plots, within the distance from the average of all plots to the standard deviation multiplied by 0.5 with relation to each of two axes in the graph of the Poincare plot.

Therefore, in the present embodiment, the degree of the distribution spread of the Poincare plot, the size, the shape, and whether the number of the plot points in the center portion is large or small, can be estimated based on the calculation result, and as a result, a psychological state or a physical state of an animal can be estimated.

### Tenth Embodiment

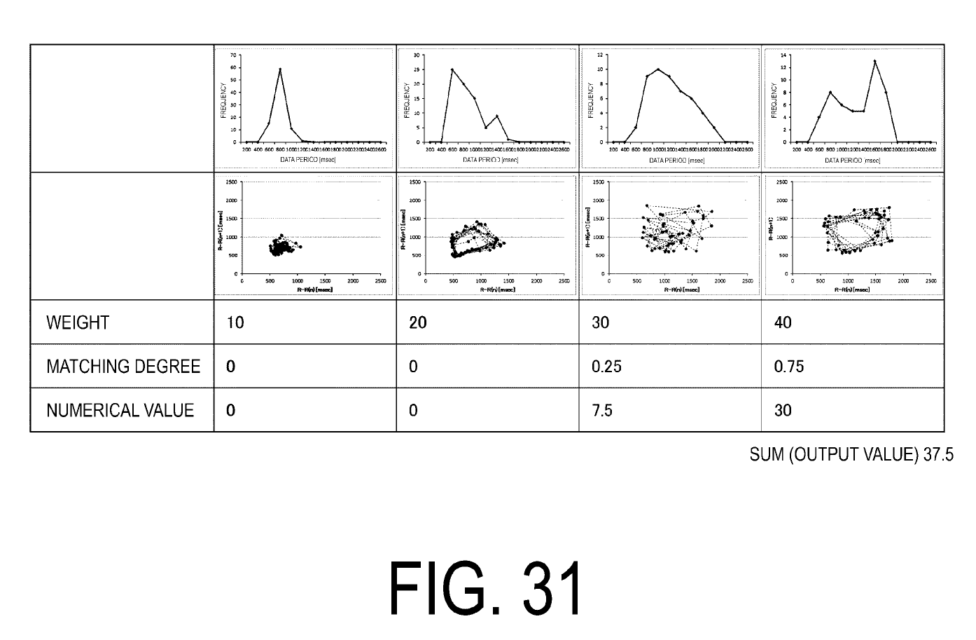

Alternatively, by using a plurality of methods among the first embodiment to the ninth embodiment, or a method different from the methods among the first embodiment to the ninth embodiment, the state of the animal may be determined. For example, as shown in FIG. 31, the state determination unit 314 may calculate the matching degree between the acquired histogram and the histogram model for each state, and determine the state based on the sum of the matching degree weighted for the respective states.

Alternatively, as shown in FIG. 31, the state determination unit 314 may calculate the matching degree between the acquired trajectory of the Poincare plot and the trajectory model of the Poincare plot for each state, and determine the state based on the sum of the matching degree weighted for the respective states.

Alternatively, as shown in FIG. 31, the state determination unit 314 may calculate the matching degree between the acquired histogram and the trajectory of the Poincare plot and the histogram model and the trajectory of the Poincare plot model for each state, and determine the state based on the sum of the matching degree weighted for the respective states.

Therefore, in the present embodiment, the degree of the distribution spread of the Poincare plot, the size, the shape, and whether the number of the plot points in the center portion is large or small, can be estimated based on the calculation result, and as a result, a psychological state or a physical state of an animal can be estimated.

### Eleventh Embodiment

Moreover, it is preferable that a user input an actual current state of the animal, and then numerical value ranges and threshold values for determining the state be revised by new data. In other words, since the user inputs a correct answer about the state due to individual difference of a dog, the CPU 310 can adjust the threshold values for the determination, and this can enhance accuracy so that the output in which the user does not recognize incongruity can be performed.

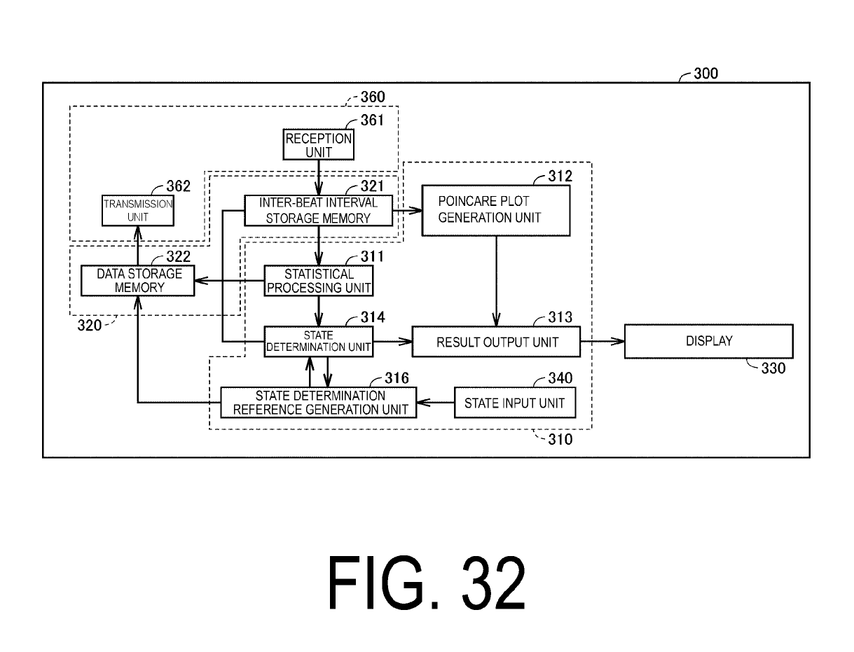

With reference to FIG. 32, a functional configuration of the state acquisition system 1 according to the present embodiment will be described below. FIG. 32 is a diagram showing the functional configuration of the state acquisition system 1 according to the present embodiment.

The communication terminal 300 according to the present embodiment includes a state determination reference generation unit 316, and a state input unit 340, compared with the communication terminal 300 according to the fifth embodiment. For example, the CPU 310 executes a program in the memory 320, so that the state determination reference generation unit 316 is realized. The state input unit 340 is realized by a switch, a keyboard, a touch panel, or the like, and delivers an operation instruction from the user to the state determination reference generation unit 316 or the like in the CPU 310.

When the user inputs a current state of the target animal to the state input unit 340, the state determination reference generation unit 316 determines whether the current state of the animal input by the user matches the determination result by the state determination unit 314 or not. In a case where the current state of the animal input by the user does not match the determination result by the state determination unit 314, the state determination reference generation unit 316 revises the numerical value ranges or the threshold values for determining the state so that the determination result by the state determination unit 314 comes close to the inputted state.

Thus, the state acquisition system 1 according to the present embodiment can enhance accuracy of the state determination.

### Twelfth Embodiment

In the first embodiment to the eleventh embodiment, although the inter-beat intervals are calculated by using the electrodes 401, 402, 403 for acquiring the electrocardilgram, a configuration is not limited thereto. For example, a pulse wave signal may be acquired by a photoelectric pulse wave type sphygmograph or pulse oxymeter, and the inter-beat interval may be calculated from the pulse wave signal. In this case, it is preferable that a measurement part of the pulse wave be a part on which the skin is exposed, such as the tongue and the ear. Additionally, a heart sound signal may be acquired by an electronic stethoscope or the like, and the inter-beat interval may be calculated from the heart wave signal. In these cases, the measurement by a method that does not use an electrode can be performed. By using a pulse wave acquisition sensor such as a microwave Doppler sensor, a pulse wave signal may be acquired and the inter-beat interval may be calculated by the pulse wave signal. For example, a configuration can be conceived in which a microwave transmission apparatus is installed on a ceiling or the like, and a pulse wave of an animal such as a dog is acquired in a non-contact manner. In this case, measurement in the non-contact manner can be performed, and has an effect in which loads on a subject are further reduced.

### Thirteenth Embodiment

In the state acquisition system 1 according to the first embodiment to the twelfth embodiment, the signal processing apparatus 500 acquires the inter-beat interval based on the electrocardiographic signal from the electrodes 401, 402, 403, and the communication terminal 300 calculates and outputs information for determining the state of the animal from the inter-beat interval or information related to the determination result about the state of the animal. However, all or part of the roles of one of these apparatuses may be played by another apparatus, or may be shared by a plurality of apparatuses. Conversely, all or part of the roles of the plurality of apparatuses may be played by an apparatus, and may be played by another apparatus.

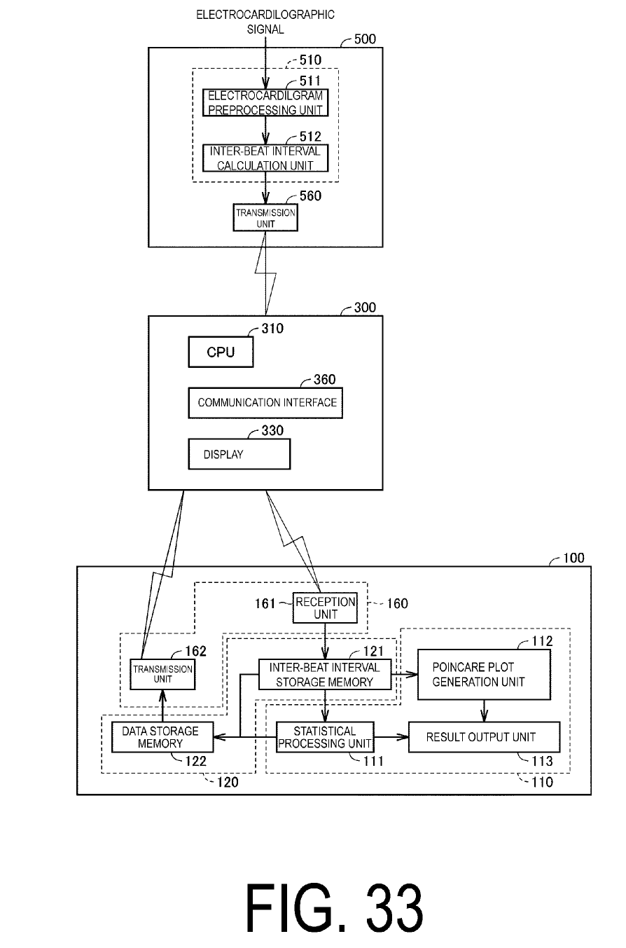

For example, as shown in FIG. 33, the server 100 may play a role of the communication terminal 300. In this case, the functions of the communication terminal 300 according to the first embodiment to the twelfth embodiment are installed in the server 100. For example, the communication terminal 300 transmits necessary information such as the inter-beat interval from the signal processing apparatus 500 to the server 100 via a router, a carrier network, the Internet or the like. The server 100 calculates information for determining the state of the animal or information indicating the determination result about the state of the animal, and transmits the information to the communication terminal 300. It is conceivable that the communication terminal 300 outputs information about a final result to a display or a speaker.

Note that, in this case, naturally, the reception unit 161 and the transmission unit 162 in the server 100 are realized by the communication interface 160 in the server 100. Additionally, the inter-beat interval storage memory 121 and the data storage memory 122 are realized by the memory 120 in the server 100 or another apparatus that can be accessed by the server 100. The CPU 110 executes a program in the memory 120, so that the statistical processing unit 111, the Poincare plot generation unit 112, and the result output unit 113 are realized.

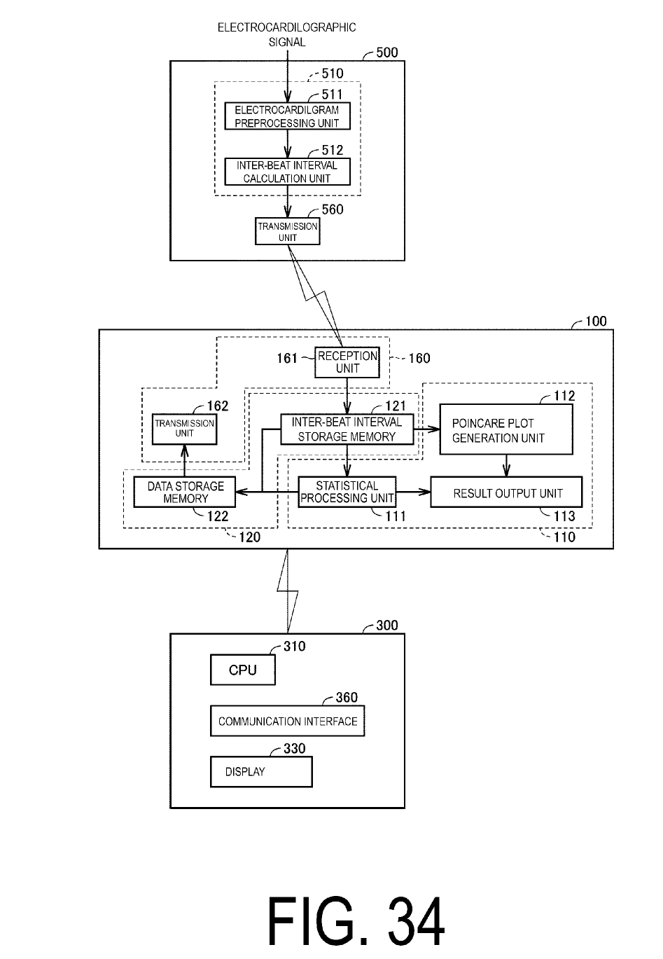

Alternatively, as shown in FIG. 34, the signal processing apparatus 500 transmits necessary information such as the inter-beat interval to the server 100 via a router, a carrier network, the Internet or the like. The server 100 calculates information for determining the state of the animal or information related to the determination result about the state of the animal, and transmits the information to the communication terminal 300 via the Internet, a router, a carrier network, or the like. The communication terminal 300 outputs information about a final result to a display or a speaker. In this case, the signal processing apparatus 500 and the communication terminal 300 do not need to be connected through a wireless LAN or a wired LAN.

Note that, in this case, naturally, the reception unit 161 and the transmission unit 162 in the server 100 are realized by the communication interface 160 in the server 100. Additionally, the inter-beat interval storage memory 121 and the data storage memory 122 are realized by the memory 120 in the server 100 or another apparatus that can be accessed by the server 100. The CPU 110 executes a program in the memory 120, so that the statistical processing unit 111, the Poincare plot generation unit 112, and the result output unit 113 are realized.

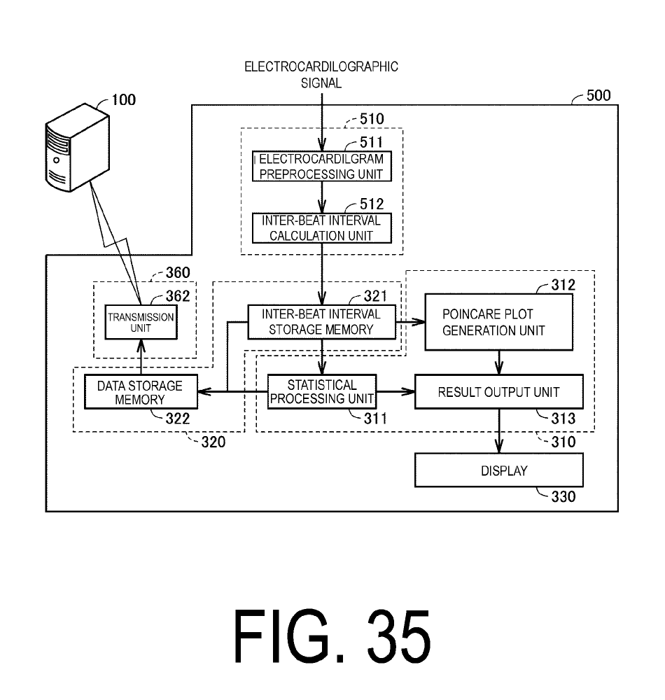

Alternatively, as shown in FIG. 35, the signal processing apparatus 500 may install all or part of the functions of the communication terminal 300. In this case, the signal processing apparatus 500, based on the electrocardiographic signal from the electrodes 401, 402, 403, calculates the information for determining the state of the animal and the inter-beat interval or the information related to the determination result about the state of the animal. Additionally, the signal processing apparatus 500 outputs the information about the final result to a display or a speaker.

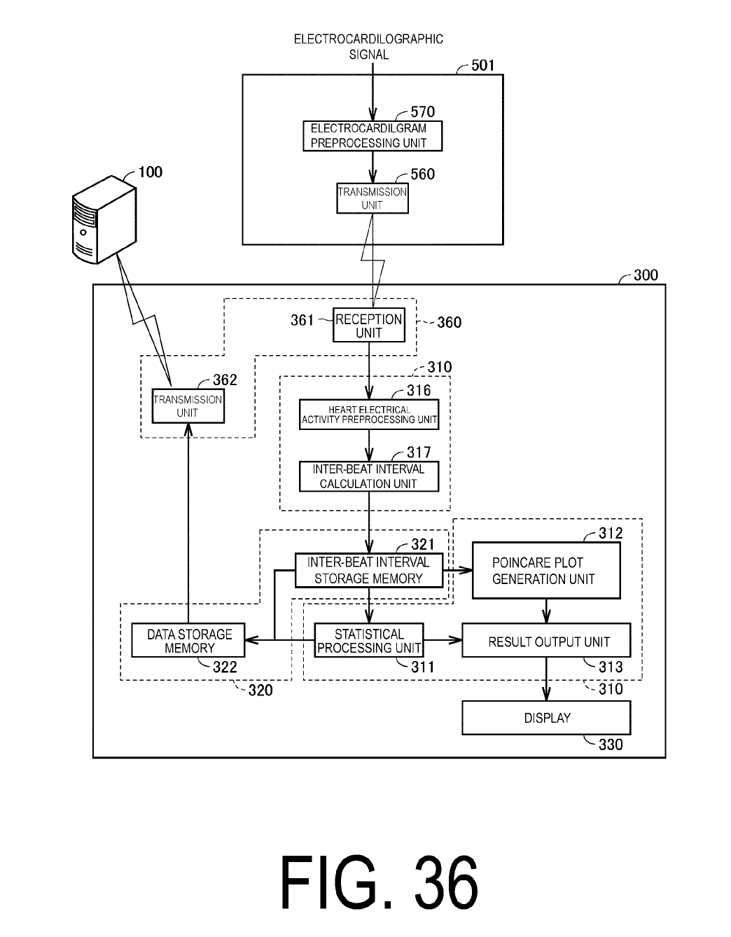

Alternatively, as shown in FIG. 36, the communication terminal 300 may install all or part of the functions of the signal processing apparatus 500. In this case, the communication terminal 300 acquires the electrocardiographic signal from the electrodes 401, 402, 403 from a simple signal processing apparatus 501 by wireless communication. The electrocardiographic signal from the electrodes is converted to a digital signal by a simple electrocardilgram preprocessing unit 570 including at minimum a filter apparatus, amplification apparatus, and A/D conversion apparatus, and is transmitted from the transmission unit 560. The communication terminal 300, from the electrocardiographic signal, calculates the information for determining the state of the animal and the inter-beat interval or the information related to the determination result about the state of the animal. Additionally, the communication terminal 300 outputs the information about the final result to a display or a speaker.

### Fourteenth Embodiment

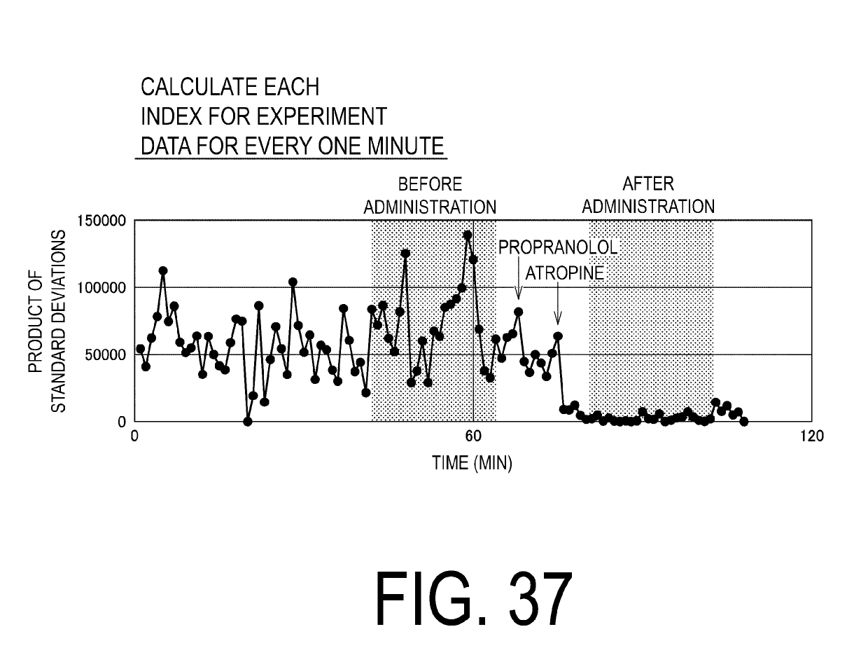

In addition to the functions of the state acquisition system 1 according to the first embodiment to the thirteenth embodiment, the state acquisition system 1 may further include the following functions. For example, as shown in FIG. 37, it is also effective to output a time series graph of the products of the standard deviations of the Poincare plot after the axis conversion. Note that FIG. 37 shows change in the product value of the standard deviations each one minute in administrating a medicine to a dog. Here, by administrating a propranolol (sympatholytic: 0.05 mg/kg BW) and an atropine (parasympatholytic: 0.1 mg/kg BW), neural activity in heat pulsations is blocked, and then the change in the product of the standard deviations is observed.

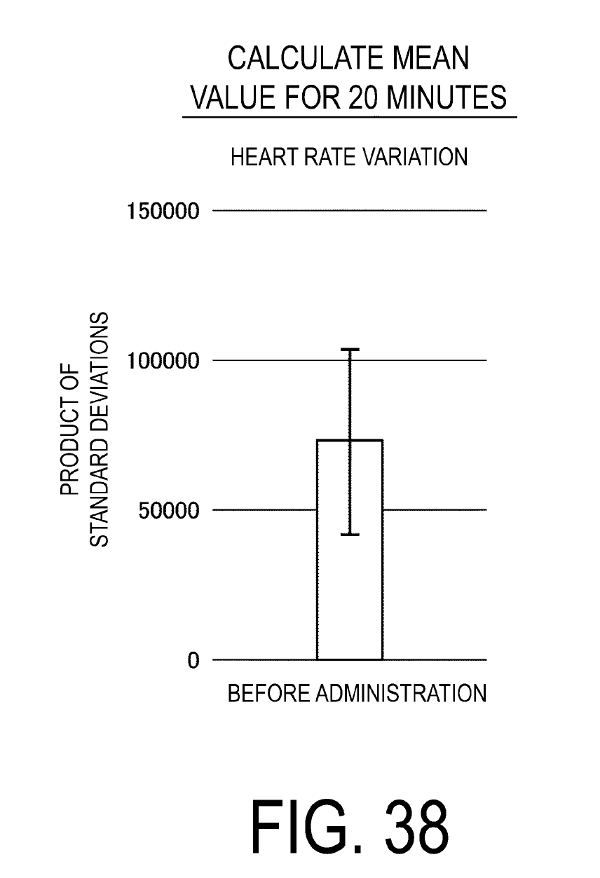

Additionally, as shown in FIG. 38, it is also effective to output a graph showing a mean value and variation (a standard deviation) of the products of the standard deviations of the Poincare plot after the axis conversion. Note that FIG. 38 is a graph showing a mean value and variation of the products of the standard deviations for 20 minutes before the above-mentioned medicine is administrated.

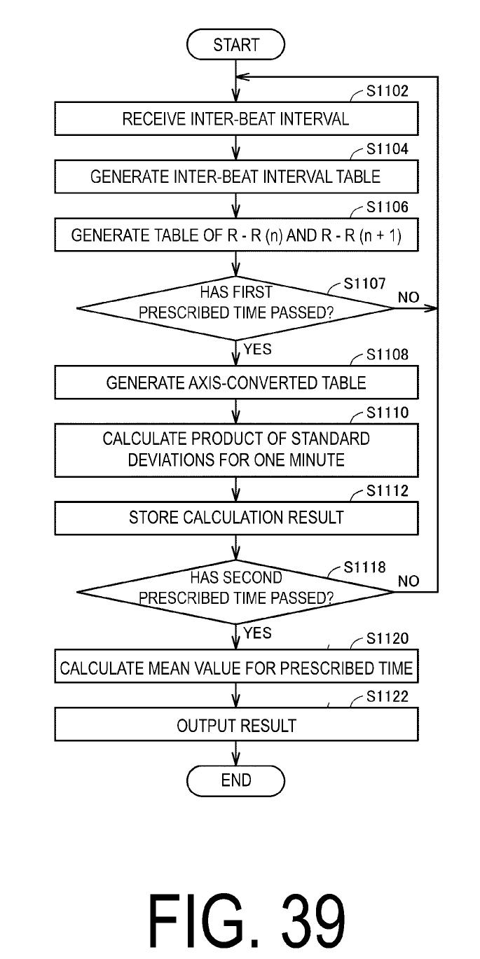

More particularly, in the present embodiment, the CPU 310 that realizes the statistical processing unit 311, the result output unit 313, and the like according to the first embodiment to the thirteenth embodiment, executes processing shown in FIG. 39. Note that, as described in the thirteenth embodiment, all or part of the processing in FIG. 39 may be executed by the communication terminal 300, the server 100, the signal processing apparatus 500, or another apparatus. Moreover, a terminal is also not limited in which information indicating a graph, a numerical value, and the like is output as in FIG. 38, FIG. 39, or the like.

Additionally, the state acquisition system 1 according to the present embodiment may or may not include the Poincare plot generation unit 312 shown in FIG. 2.

With reference to FIG. 39, the reception unit 361 receives data indicating the inter-beat interval from the signal processing apparatus 500 (step S1102).

The inter-beat interval storage memory 321 is formed of various types of memories 320 and stores the data received from the signal processing apparatus 500. In the present embodiment, the CPU 310 sequentially stores the inter-beat intervals received via the communication interface 360 as an inter-beat interval table in the memory 320 (step S1104). However, these data may be stored in the memory 320 of the communication terminal 300, or in other apparatuses that can be accessed from the communication terminal 300.

For example, the CPU 310 executes a program in the memory 320, so that the statistical processing unit 311, and the result output unit 313 are realized. The statistical processing unit 311 reads the inter-beat interval data from the inter-beat interval storage memory 321 at a fixed time unit, and generates the table 321A indicating correspondence relation between an inter-beat interval R-R (n) and the following inter-beat interval R-R (n+1), as shown in FIG. 4 (step S1106).

As shown in FIG. 5, after a first prescribed time, for example one minute, or 10 minutes, passes (in a case of YES in a step S1107), the statistical processing unit 311 converts the table indicating correspondence relation between the inter-beat interval R-R (n) and the following inter-beat interval R-R (n+1) into axes in a Y=X direction and a direction perpendicular to the Y=X direction (step S1108).

The statistical processing unit 311 calculates a standard deviation and a product of the standard deviations related to numerical series forming each axis after the axis conversion (step S1110). Note that the statistical processing unit 311 may calculate only a standard deviation related to a Y=X axis, only a standard deviation related to an axis perpendicular to the Y=X axis, or both the standard deviations.

The statistical processing unit 311 stores the product of the standard deviations this time after the axis conversion in the storage memory 322 (step S1112). The statistical processing unit 311, in a case where a second prescribed time, for example, 20 minutes, or 24 hours, has not passed (in a case of NO in a step S1118), repeats the processing from the step S1102.

The statistical processing unit 311, in a case where the second prescribed time has passed (in a case of YES in the step S1118), calculates a mean value and variation (standard deviation) of the products of the standard deviations for the second prescribed time (step S1120).

The result output unit 313 allows an output device of itself or an external output device, such as the display 330 or a speaker, to display a result image, text, or output an audio message (step S1122). More particularly, the result output unit 313, based on the stored data in the step 1112, outputs and renews a time series graph of the products of the standard deviations of the Poincare plot after the axis conversion, as shown in FIG. 37. In addition, the result output unit 313, based on the calculation result in the step S1120, outputs and renews a graph showing the mean value and variation of the products of the standard deviations, as shown in FIG. 38.

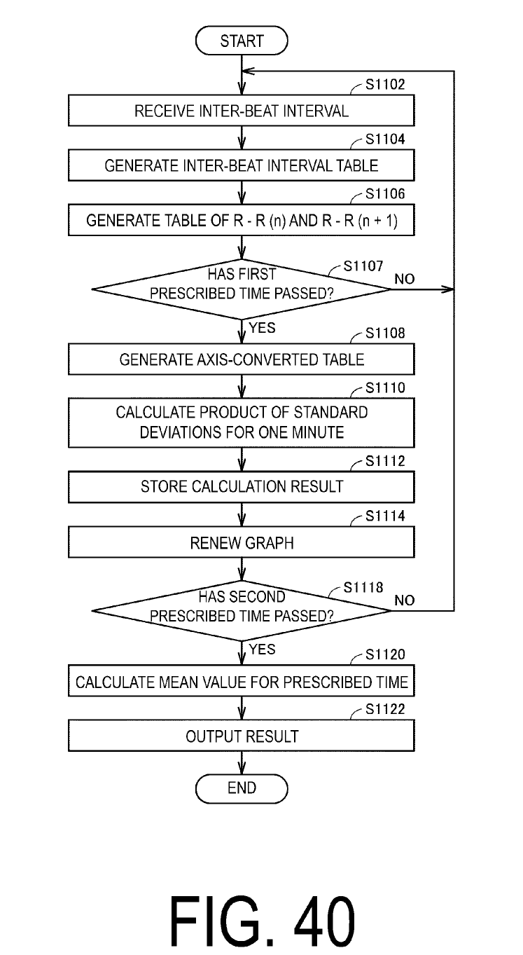

More particularly, as shown in FIG. 40, after the step 1112, that is, for each first prescribed time, the result output unit 313 may output and renew the time series graph of the products of the standard deviations of the Poincare plot after the axis conversion as shown in FIG. 37 (step S1114).

### Fifteenth Embodiment

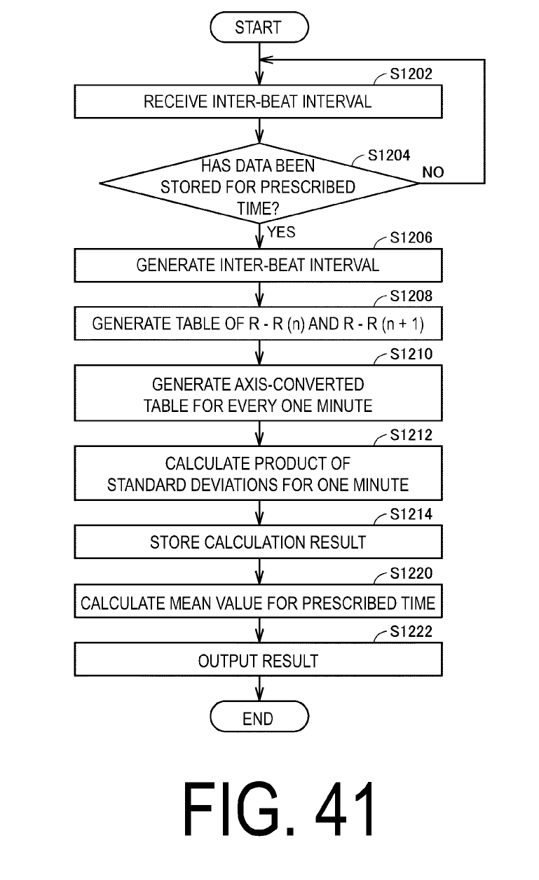

Alternatively, the CPU 310 may execute the processing shown in FIG. 41. In other words, the reception unit 361 receives data indicating the inter-beat interval from the signal processing apparatus 500 (step S1202).

The inter-beat interval storage memory 321 is formed of various types of memories 320 and stores the data received from the signal processing apparatus 500. In the present embodiment, in a case where the first prescribed time, for example, 20 minutes, an hour, or 24 hours, has passed (in a case of YES in a step S1204) the CPU 310 sequentially stores the inter-beat intervals received via the communication interface 360 as an inter-beat interval table in the memory 320 (step S1206). However, these data may be stored in the memory 320 of the communication terminal 300, or in other apparatuses that can be accessed from the communication terminal 300.

The statistical processing unit 311 reads the inter-beat interval data from the inter-beat interval storage memory 321 at a fixed time unit, and generates the table 321A indicating correspondence relation between an inter-beat interval R-R (n) and the following inter-beat interval R-R (n+1), as shown in FIG. 4 (step S1208).

As shown in FIG. 5, every second prescribed time, for example, every one minute, or 10 minutes, the statistical processing unit 311 converts the table indicating correspondence relation between the inter-beat interval R-R (n) and the following inter-beat interval R-R (n+1) into axes in a Y=X direction and a direction perpendicular to the Y=X direction (step S1210).

The statistical processing unit 311 calculates a standard deviation and a product of the standard deviations related to numerical series forming each axis after the axis conversion (step S1212). The statistical processing unit 311 stores the product of the standard deviations this time after the axis conversion in the storage memory 322 (step S1214).

The statistical processing unit 311, calculates a mean value and variation (standard deviation) of the products of the standard deviations for the first prescribed time (step S1220).

The result output unit 313 allows, for example, an output device of itself or an external output device, such as the display 330 or a speaker, to output an image, text or an audio message related to a result (step S1222). More particularly, the result output unit 313, based on the stored data in the step 1112, outputs and renews a time series graph of the products of the standard deviations of the Poincare plot after the axis conversion as shown in FIG. 37. In addition, the result output unit 313, based on the calculation result in the step S1120, outputs and renews a graph showing the mean value and variation of the products of the standard deviations, as shown in FIG. 38.

### Sixteenth Embodiment

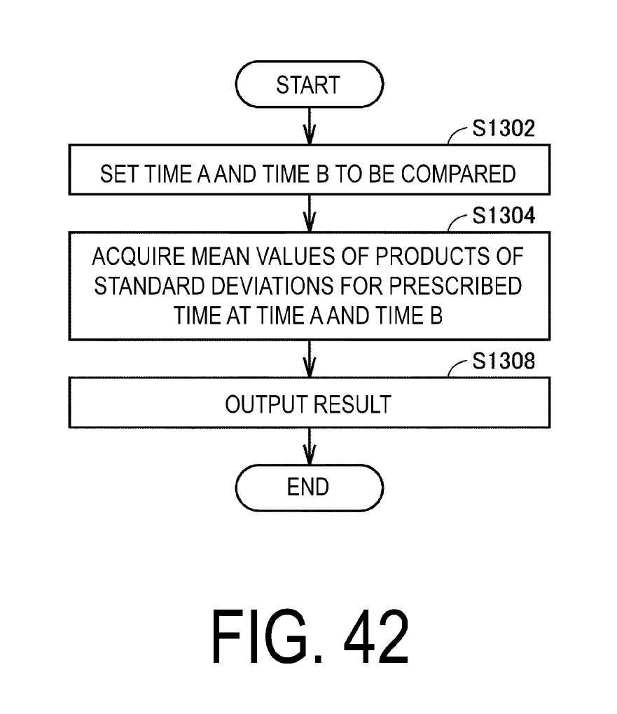

Further, the statistical processing unit 311 may execute processing shown in FIG. 42 after the step S1120 and the step S1220. With reference to FIG. 42, the statistical processing unit 311 accepts settings at time A and time B that are compared with each other, for example, from the server 100, a smartphone, or the like (step S1302). The statistical processing unit 311 reads the mean values of the products of the standard deviations for the prescribed time at the time A and the time B calculated in the step S1120 and the step S1220 (step S1304).

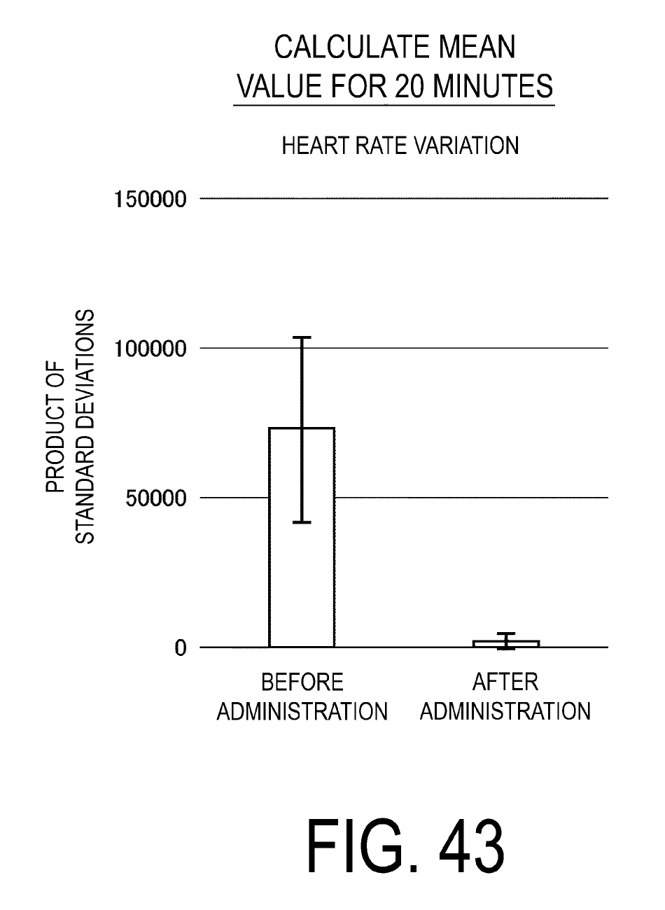

The result output unit 313 allows, for example, an output device of itself or an external output device, such as the display 330 or a speaker, to output an image, text or an audio message related to a result (step S1308). For example, the result output unit 313, as shown in FIG. 43, outputs a graph showing a mean value of the stored data of products of standard deviations every one minute for 20 minutes and variation of products of standard deviations, before and after administration of medicine.

FIG. 43 is a diagram showing a result of the mean values calculated for every 20 minutes, except for five minutes before and after the administration of the medicine (because fluctuation occurs due to the administration for five minutes before and after the administration of the medicine), as shown in FIG. 37. Compared with before the administration, the variation of the inter-beat intervals is reduced, and the product of the standard deviations becomes smaller because of the medicine after the administration. Averaging the variation of the standard deviations makes it possible to easily compare magnitude of time series change in variation of the inter-beat intervals.

Here, although the example is described in which specific time is designated as comparison targets, the comparison target is not limited thereto. For example, data acquired in any term and any time period may be averaged and compared, such as comparison of a mean value of data every eight o'clock in the morning and a mean value of data every eight o'clock at night for a week, and comparison of a mean value of data for a month and a mean value of data for another month. Additionally, the comparison targets are not limited to two terms, and every prescribed time, for example, every two hours, a mean value for the prescribed time may be calculated, and the transition may be output as a graph.

### Seventeenth Embodiment

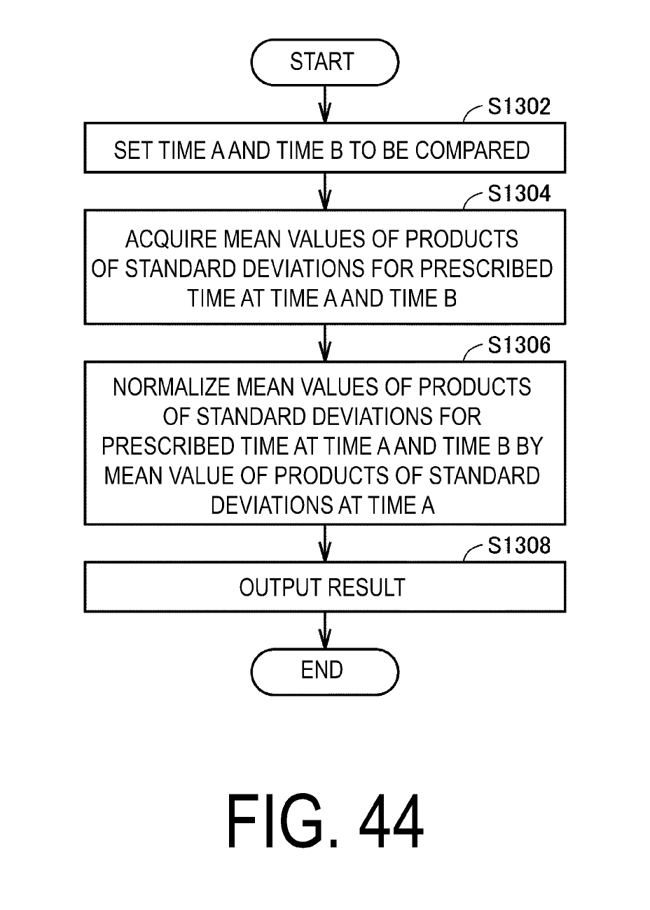

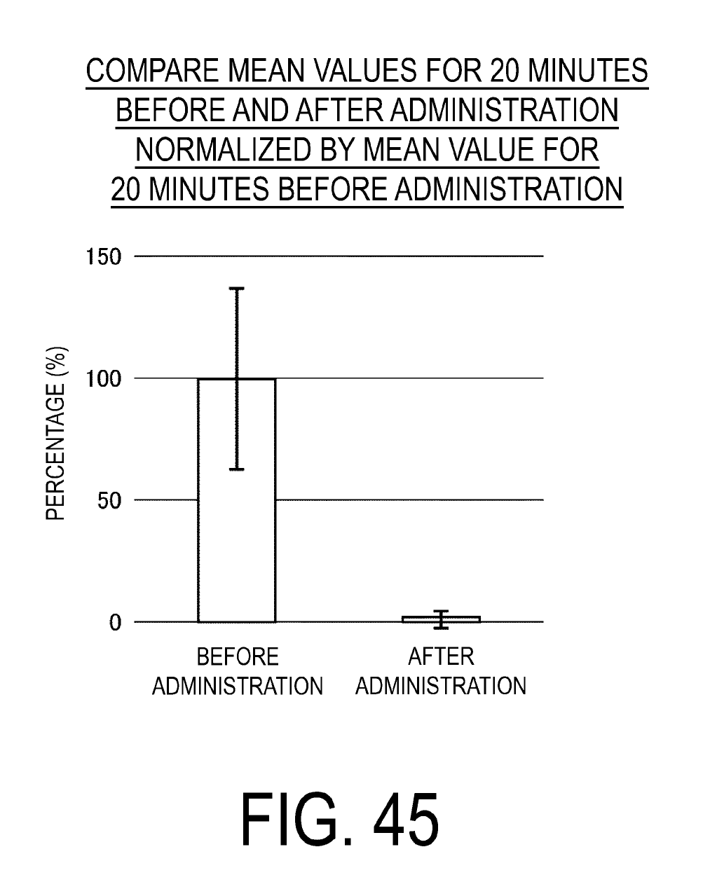

Further, the statistical processing unit 311 may execute processing shown in FIG. 44 after the step S1120 and the step S1220. With reference to FIG. 44, the statistical processing unit 311, after the step S1304, may normalize the mean values of the products of the standard deviations for the prescribed time at the time A and the time B by the mean value of the products of the standard deviations for the prescribed time at the time A (step S1306). Moreover, the result output unit 313, as shown in FIG. 45, preferably outputs the normalized graphs of the products of the standard deviations of the inter-beat intervals before and after the administration of the medicine (step S1308).

This can reduce variation due to an individual difference or time, and compare a change rate of numerical values before and after the prescribed time.

### Supplementary Description of Fourteenth Embodiment to Seventeenth Embodiment

As shown in FIG. 37, the products of the standard deviations of the Poincare plot after the axis conversion are targeted in the fourteenth embodiment to the seventeenth embodiment. However, numerical values to be targeted are not limited thereto, the products of the standard deviations of the Poincare plot without the axis conversion may be used, any standard deviation may be simply used, or a distance between two Poincare plots may be used.

### About Terms

In the above descriptions, although the processing for performing the “Poincare plot” and the processing for performing the “axis conversion after the Poincare plot processing” are described, these processes should not be limited to cases where the CPU in each of the communication terminal 300, the server 100, and the signal processing apparatus 500 actually prints or displays a Poincare plot image in a paper medium or a display. These processes have a concept including processing in which a CPU stores and expands data substantially indicating the Poincare plot in a memory. Additionally, although the “Poincare plot” is also referred to as a “Lorenz plot”, the Poincare plot means plotting on two orthogonal axes with a point at a time n and a point at the following time n+1 made as the respective axes, and is not restricted by the term “Poincare plot.”

### Other Applications

Needless to say, this disclosure can be adapted in a case where this disclosure is achieved by supplying a program to a system or an apparatus. Moreover, the effect of this disclosure can be achieved by supplying a storage medium (or memory) that stores a program expressed by a software for achieving this disclosure to a system or an apparatus so that a computer (or a CPU or MPU) in the system or the apparatus reads and executes program codes stored in the storage medium.

In this case, the program codes themselves read from the storage medium enable the functions of the embodiments, and the storage medium that stores the program codes configures this disclosure.

Moreover, needless to say, cases are included in which not only the functions of the above-described embodiments are enabled by executing the program codes read by the computer, but also, based on instructions of the program codes, an Operating System (OS) or the like operating on the computer performs all or part of actual processing, and by the processing, the above-described embodiments are enabled.

Furthermore, needless to say, cases are included in which after the program codes read from the storage medium are written in a feature expansion board inserted in a computer or another storage medium included in a feature expansion unit connected to a computer, based on instructions of the program codes, a CPU or the like included in the feature expansion board or the feature expansion unit performs all or part of actual processing, and by the processing, the above-described embodiments are enabled.

The embodiments disclosed here are to be understood as being in all ways exemplary and in no ways limiting. The scope of the disclosure is defined not by the foregoing descriptions but by the appended claims, and is intended to include all changes equivalent in meaning and scope to the claims.

### REFERENCE SIGNS LIST

1: State acquisition system
100: State acquisition computer (server)
110: Processor (CPU)
111: Statistical processing unit
112: Poincare plot generation unit
113: Result output unit
120: Memory
121: Inter-beat interval storage memory
122: Data storage memory
160: Communication interface
161: Reception unit
162: Transmission unit
300: State acquisition computer (communication terminal)
310: Processor (CPU)
311: Statistical processing unit
312: Poincare plot generation unit
313: Result output unit
314: State determination unit
315: Histogram generation unit
316: State determination reference generation unit
320: Memory
321: Inter-beat interval storage memory
321A: Correspondence relation table
321B: Distance table
322: Data storage memory
330: Display (output device)
340: State input unit
360: Communication interface
361: Reception unit
362: Transmission unit
401: Sensor (electrode)
402: Sensor (electrode)
403: Sensor (electrode)
500: Signal processing apparatus
501: Simple signal processing apparatus
510: Processor (CPU)
511: Electrocardilgram preprocessing unit
512: Inter-beat interval calculation unit
560: Transmission unit
570: Simple electrocardilgram preprocessing unit

1-15. (canceled)

16. A state acquisition computer comprising:
an interface configured to acquire data for indicating an inter-beat interval of an animal; and
a processor configured to acquire information indicating a psychological state or a physical state of the animal, based on a degree of proximity between plots in a Poincare plot of inter-beat intervals.

17. The state acquisition computer according to claim 16,
wherein the processor is configured to acquire the information indicating the psychological state or the physical state of the animal, based on a product of standard deviations of at least two axes of the Poincare plot.

18. The state acquisition computer according to claim 16,
wherein the processor is configured to acquire the information indicating the psychological state or the physical state of the animal, based on an average of distances from one plot to another plot that are two successive plots in the Poincare plot of inter-beat intervals.

19. The state acquisition computer according to claim 17, further comprising:
a memory configured to store a numerical value range corresponding to each of a plurality of types of psychological states or physical states,
wherein the processor is configured to identify a psychological state or a physical state of the animal by referring to the numerical value range, based on the standard deviation.

20. The state acquisition computer according to claim 16,
wherein an animal being a target has a respiratory sinus arrhythmia.

21. A state acquisition method of a psychological state or a physical state of an animal, the method being performed on a computer including a processor, comprising:
acquiring data for indicating an inter-beat interval of the animal;
calculating a degree of proximity between plots in a Poincare plot of inter-beat intervals; and
acquiring information indicating the psychological state or the physical state of the animal, based on the degree of proximity.

22. The state acquisition method according to claim 21,
wherein the degree of proximity is calculated based on an average of distances from one plot to another plot that are two successive plots in the Poincare plot of inter-beat intervals.

23. An information processing system comprising:
an output device;
a sensor configured to detect a beat of an animal; and
a computer configured to acquire data for indicating an inter-beat interval of the animal from the sensor, to acquire information indicating a psychological state or a physical state of the animal based on a degree of proximity between plots in a Poincare plot of inter-beat intervals, and to cause the output device to output the information.

24. The state acquisition computer according to claim 18, further comprising:
a memory configured to store a numerical value range corresponding to each of a plurality of types of psychological states or physical states,
wherein the processor is configured to identify a psychological state or a physical state of the animal by referring to the numerical value range, based on the average.

25. The state acquisition method according to claim 21,
wherein the degree of proximity is calculated based on a product of standard deviations of at least two axes of the Poincare plot.

26. The information processing system according to claim 23,
wherein the computer is configured to acquire the information indicating the psychological state or the physical state of the animal, based on a product of standard deviations of at least two axes of the Poincare plot.

27. The information processing system according to claim 23,
wherein the computer is configured to acquire the information indicating the psychological state or the physical state of the animal, based on an average of distances from one plot to another plot that are two successive plots in the Poincare plot of inter-beat intervals.

28. The state acquisition computer according to claim 16,
wherein the processor is configured to acquire the information indicating the psychological state or the physical state of the animal, based on a product of standard deviations along a Y=X axis or a product of standard deviations along an axis perpendicular to the Y=X axis of the Poincare plot of inter-beat intervals.

29. The state acquisition computer according to claim 16,
wherein the processor is configured to specify an axis along which a variance of the Poincare plot of inter-beat intervals is maximized and to calculate standard deviations with respect to the axis and an axis perpendicular to the axis.

30. The information processing system according to claim 23, further comprising:
a control program configured to cause the computer to function; and
a non-transitory computer readable medium in which the control program is stored.
